(19) United States
(12) Patent Application Publication
(10) Pub. No.: US 2023/0184770 A1
(43) Pub. Date: Jun. 15, 2023

(54) Exosome Analysis And Brain Tumors

(71) Applicant: Institute For Cancer Research d/b/a The Research Institute of Fox Chase Cancer Center, Philadelphia, PA (US)

(72) Inventors: Zengjie Yang, Philadelphia, PA (US); Yijung Yang, Philadelphia, PA (US)

(21) Appl. No.: 17/920,356

(22) PCT Filed: Apr. 21, 2021
(86) PCT No.: PCT/US21/28431

§ 371 Date: Oct. 20, 2022

Related U.S. Application Data
(60) Provisional application No. 63/013,800, filed on Apr. 22, 2020.

(51) Int. Cl. G01N 33/574; G01N 33/50

(52) U.S. Cl. CPC G01N 33/57407; G01N 33/5076

(57) ABSTRACT

The present disclosure provides methods of diagnosing, preventing, monitoring, and treating brain tumors. In particular, the present disclosure provides methods of using brain tum or biomarkers in exosomes for diagnosing, preventing, monitoring, and treating brain tumors.

### REFERENCE TO GOVERNMENT GRANTS

This invention was made with government support under Grant No. CA178380 and Grant No. CA185504 awarded by the National Institutes of Health. The government has certain rights in the invention.

### FIELD

The present disclosure is directed, in part, to methods of diagnosing, preventing, monitoring, and treating brain tumors in a subject. In particular, the present disclosure is directed to using brain tumor biomarkers present in an exosome in such methods.

### BACKGROUND

Cancers of the brain and nervous system are among the most difficult to treat. Prognosis for patients with these cancers depends on the type and location of the tumor as well as its stage of development. The classification of brain tumors is associated with the cell type from which they arise. Astrocytes, oligodendrocytes, and glial cells may give rise to brain tumors. The incidence of brain tumor is estimated to be 22.64 per 100,000 persons in the United States. In addition, approximately one third of brain tumors are malignant. Medulloblastoma (MB) and glioblastoma multiforme (GBM) represent the most common malignant brain tumor in children and adults, respectively.

For many types of brain cancer, the average life expectancy after symptom onset may be from months to a year or two. Treatment consists primarily of surgical removal and radiation therapy. Chemotherapy is also used, but the range of suitable chemotherapeutic agents is limited, perhaps because most therapeutic agents do not penetrate the blood-brain barrier adequately to treat brain tumors. Using known chemotherapeutic agents along with surgery and radiation can, although rarely, extend survival much beyond that produced by surgery and radiation alone. Thus, improved therapeutic options are needed for brain tumors.

An additional challenge in the management of brain tumors lies in the difficulty of obtaining tissue samples. Brain tumor samples can only be obtained by surgical resection or biopsy. Such invasive procedures are not routine clinical practice due to possible life-threatening complications and the intracerebral location. Non- or minimally-invasive methods for brain tumor diagnosis, as well as monitoring brain tumor progression and therapeutic responsiveness, are needed.

Exosomes are small membrane vesicles that are released from many cell types into the extracellular environment. Although, microvesicles and exosomes were initially thought to be products of a pathway used to release excess material from cells, they have been shown to mediate morphogen signaling, immunological signaling, cell recruitment, and horizontal transfer of genetic material. Exosomes are derived from the luminal membranes of late endosomes/multivesicular bodies (MVB), and are constitutively released via the fusion of MVBs with the cell membrane.

### SUMMARY

The present disclosure provides methods of identifying a human having a brain tumor, the methods comprising: assaying the level of one or more brain tumor biomarkers in an exosomal sample obtained from the human; comparing the level of the one or more brain tumor biomarkers in the exosomal sample from the human to the levels of the corresponding one or more brain tumor biomarkers in a reference exosomal sample, wherein an increase in the level of the one or more brain tumor biomarkers in the exosomal sample from the human compared to the reference exosomal sample indicates the presence of a sub-type of a brain tumor in the human; and administering an anti-exosome therapeutic agent to the human.

The present disclosure also provides methods of classifying a medulloblastoma tumor in a human, the methods comprising: assaying the level of one or more medulloblastoma biomarkers chosen from CTTNB1, DKK1, WIF1, TNC, GAD1, DDK2, EMX2, ATOH1, EYA1, HHIP, PDLIM3, SFRP1, NPR3, IMPG2, GABRA5, EGFL11, MAB21L2, KCNA1, EOMES, KHDRBS2, RBM24, UNC5D, and OAS1, in an exosomal sample obtained from the human; comparing the level of the one or more biomarkers in the exosomal sample from the human to the levels of the corresponding one or more biomarkers in a reference exosomal sample, wherein: an increase in the level of one or more of ATOH1, EYA1, HHIP, PDLIM3, and SFRP1 in the exosomal sample from the human compared to the reference exosomal sample indicates the presence of a sonic hedgehog subgroup medulloblastoma; an increase in the level of one or more of CTTNB1, DKK1, WIF1, TNC, GAD1, DDK2, and EMX2 in the exosomal sample from the human compared to the reference exosomal sample indicates the presence of a Wnt subgroup medulloblastoma; an increase in the level of one or more of NPR3, IMPG2, GABRA5, EGFL11, and MAB21L2 in the exosomal sample from the human compared to the reference exosomal sample indicates the presence of a Group C medulloblastoma; and an increase in the level of one or more of KCNA1, EOMES, KHDRBS2, RBM24, UNC5D, and OAS1 in the exosomal sample from the human compared to the reference exosomal sample indicates the presence of a Group D medulloblastoma; and administering an anti-exosome therapeutic agent to the human.

The present disclosure also provides methods of treating a human having a brain tumor comprising administering to the human in need thereof an anti-exosome therapeutic agent.

The present disclosure also provides methods of suppressing vismodegib resistance in a human having a vismodegib-resistant brain tumor, the methods comprising administering to the human in need thereof an anti-exosome therapeutic agent.

The present disclosure also provides methods of monitoring brain tumor treatment in a human comprising: assaying the level of one or more brain tumor biomarkers in a first exosomal sample obtained from the human and a second exosomal sample obtained from the human, wherein the second exosomal sample is obtained from the human after the first exosomal sample; and comparing the level of the one or more brain tumor biomarkers in the first exosomal sample to the level of the one or more brain tumor biomarkers in the second exosomal sample, wherein: a decrease in the level of the one or more brain tumor biomarkers in the second exosomal sample compared to the first exosomal sample indicates the human is responding favorably to the brain tumor treatment; and no change or an increase in the level of the one or more brain tumor biomarkers in the second exosomal sample compared to the first exosomal sample indicates the human is not responding favorably to the brain tumor treatment.

The present disclosure also provides anti-exosome therapeutic agents for use in treating a human having a brain tumor.

The present disclosure also provides anti-exosome therapeutic agents for use in the preparation of a medicament for treating a human having a brain tumor.

The present disclosure also provides anti-exosome therapeutic agents for use in suppressing vismodegib resistance in a human having a vismodegib-resistant brain tumor.

The present disclosure also provides anti-exosome therapeutic agents for use in the preparation of a medicament for suppressing vismodegib resistance in a human having a vismodegib-resistant brain tumor.

### BRIEF DESCRIPTION OF THE DRAWINGS

FIG. 1 shows the results of a Nanosight assay measuring particle numbers and size of exosomes isolated from plasma of Math1-Cre/Rosa-GFP/Ptch1−/− mice.

FIG. 2A shows MB cells express GFP in a Math1-Cre/Rosa-GFP/Ptch1−/− mouse (A, arrow points to the MB).

FIG. 2B shows detection of GFP protein in the plasma of two Math1-Cre/Rosa-GFP/Ptch1−/− mice (#1 and #2); CD63 protein was used as a loading control.

FIG. 3 shows levels of mRNA specific for Hh MB in the plasma from wild type mice or Ptch1−/− mice determined by q-PCR.

FIG. 4 shows Ki67 immunocytochemical assay of MB cells treated with DMSO, vismodegib (vismo) or vismodegib together with exosomes from vismodegib treated MB cells (vismo+Exo); DAPI was used to counterstain cell nuclei.

FIG. 5 shows the percentage of Ki67⁺ cells in MB cells after the treatment as shown in FIG. 4.

FIG. 6 shows levels of Gli1 mRNA in MB cells examined by q-PCR after the treatment as shown in FIG. 4.

FIG. 7 shows Western blotting assay of Ras levels in exosomes from MB cells treated with DMSO or vismodegib; CD63 protein expression was used as a loading control.

FIG. 8. shows the synergistic effect of vismodegib and GW4869 in inhibition of MB cell proliferation.

FIG. 9A shows the outline of an exosome secretion inhibition assay.

FIG. 9B shows results of a bioluminescence assay of tumor growth in mice injected with MB cells and treated with vismodegib±GW4869.

FIG. 9C shows a summary of the tumor growth bioluminescence assay.

### DESCRIPTION OF EMBODIMENTS

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the phrase “brain tumor” refers to the presence of abnormal cells within the brain or any other tissue within the nervous system. Brian tumors include malignant or cancerous tumors, and benign tumors, as well as low grade-tumors or high-grade tumors. Cancerous tumors can be divided into primary tumors that started within the brain and those that spread from somewhere else known as brain metastasis tumors. The most common primary brain tumors are gliomas, meningiomas, pituitary adenomas, and nerve sheath tumors. Brain tumors also include glioblastomas and medulloblastomas, including ssh subgroup, Wnt subgroup, Group 3 subgroup, and Group 4 subgroup.

As used herein, the term “comprising” may be replaced with “consisting” or “consisting essentially of” in particular embodiments as desired.

As used herein, the phrase “level of brain tumor biomarker” and the like encompasses the type of brain tumor biomarker and/or the amount of brain tumor biomarker present in an exosome. In some embodiments, the level of one or more brain tumor biomarkers includes a number of different exosome measurements including, but not limited to, the total number of exosomes containing a brain tumor biomarker (e.g., total exosomes per mL plasma), total exosome brain tumor biomarker protein (i.e., total level of brain tumor biomarker protein per exosome or total exosome level of brain tumor biomarker protein per mL of patient plasma), total exosome brain tumor biomarker DNA or RNA (i.e., total level of brain tumor biomarker DNA and/or RNA per exosome or total exosome level of brain tumor biomarker DNA and/or RNA per mL of patient plasma), and/or total exosome brain tumor biomarker miRNA, or any combination thereof in a sample. Exosomes derived from brain tumors can have unique molecular signatures based on the origin of the primary tumor that can be used to diagnose the brain tumor subtype. This unique molecular signature is based on brain tumor biomarker protein, DNA, RNA, and/or microRNA content in the exosome. This method of diagnosing brain tumors is suitable for diagnosing any brain tumor including, but not limited to, glioblastoma or medulloblastoma, including sonic hedgehog subgroup medulloblastoma, Wnt subgroup medulloblastoma, group 3 medulloblastoma (also termed group C medulloblastoma), and group 4 medulloblastoma (also termed group D medulloblastoma).

As used herein, the terms “subject” and “patient” are used interchangeably. A subject may include any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human.

According to the present disclosure, tumor cell-derived exosomes were detected in plasma of mice bearing medulloblastoma tumors. In addition, exosomes secreted by mouse medulloblastoma cells were found to carry subgroup-specific mRNAs. For example, the plasma exosomes extracted from mice having sonic hedgehog (ssh) subgroup medulloblastoma were found to have elevated mRNA levels of 5 signature genes. In addition, exosomes conferred the resistance of tumor cells to vismodegib (the first FDA-approved hedgehog pathway inhibitor) by activation of Ras/MAPK signaling. Moreover, inhibition of exosome secretion significantly repressed medulloblastoma cell proliferation. Thus, suppression of exosome signaling by brain tumor cells may inhibit cell proliferation in brain tumors and overcome tumor resistance to suppressors of hedgehog pathway signaling. Accordingly, the present disclosure provides methods of leveraging the analysis of exosomes to diagnose subjects as having a brain tumor and to classify the brain tumor if found in a subject. Additionally, the present disclosure provides methods of suppressing brain tumor cell proliferation through suppressing exosome secretion, and of overcoming exosome-mediated resistance to anti-tumor agents. Furthermore, the present disclosure provides methods of leveraging the analysis of the content of serum exosomes to monitor the effectiveness of anticancer therapy administered to a brain tumor patient.

Symptoms of brain tumor include new onset or change in pattern of headaches, headaches that gradually become more frequent and more severe, unexplained nausea or vomiting, vision problems, such as blurred vision, double vision or loss of peripheral vision, gradual loss of sensation or movement in an arm or a leg, difficulty with balance, speech difficulties, confusion in everyday matters, personality or behavior changes, seizures, especially in someone who doesn't have a history of seizures, and hearing problems, or any combination thereof.

The present disclosure provides methods of identifying a human having a brain tumor. The methods comprise assaying the level of one or more brain tumor biomarkers in an exosomal sample obtained from the human. The methods also comprise comparing the level of the one or more brain tumor biomarkers in the exosomal sample from the human to the levels of the corresponding one or more brain tumor biomarkers in a reference exosomal sample. An increase in the level of the one or more brain tumor biomarkers in the exosomal sample from the human compared to the reference exosomal sample indicates the presence of a sub-type of a brain tumor in the human. In some embodiments, the methods also comprise administering an anti-exosome therapeutic agent to the human.

In some embodiments, the brain tumor is a glioblastoma. In some embodiments, the brain tumor is a medulloblastoma.

In some embodiments, the brain tumor is a Wingless (Wnt) subgroup brain tumor, a sonic hedgehog (shh) subgroup brain tumor, a Group 3 subgroup brain tumor, or a Group 4 subgroup brain tumor. In some embodiments, the brain tumor is a Wnt subgroup brain tumor. In some embodiments, the brain tumor is an shh subgroup brain tumor. In some embodiments, the brain tumor is a Group 3 subgroup brain tumor. In some embodiments, the brain tumor is a Group 4 subgroup brain tumor.

In some embodiments, the brain tumor biomarker is chosen from Catenin Beta 1 (CTTNB1), Dickkopf WNT Signaling Pathway Inhibitor 1 (DKK1), Wnt Inhibitory Factor 1 (WIF1), Tenascin C (TNC), Glutamate Decarboxylase 1 (GAD1), Dickkopf WNT Signaling Pathway Inhibitor 2 (DDK2), and Empty Spiracles Homeobox 2 (EMX2), or any combination thereof. In some embodiments, the brain tumor biomarker is chosen from WIF1, TNC, GAD1, DDK2, and EMX2, or any combination thereof. In some embodiments, the brain tumor biomarker is CTTNB1. In some embodiments, the brain tumor biomarker is DKK1. In some embodiments, the brain tumor biomarker is WIF1. In some embodiments, the brain tumor biomarker is TNC. In some embodiments, the brain tumor biomarker is GAD1. In some embodiments, the brain tumor biomarker is DDK2. In some embodiments, the brain tumor biomarker is EMX2. In some embodiments, the brain tumor is a Wnt subgroup brain tumor.

In some embodiments, the brain tumor biomarker is chosen from Atonal BHLH Transcription Factor 1 (ATOH1), EYA Transcriptional Coactivator and Phosphatase 1 (EYA1), Hedgehog-Interacting Protein (HHIP), PDZ and LIM Domain Protein 3 (PDLIM3), and Secreted Frizzled-Related Protein 1 (SFRP1). In some embodiments, the brain tumor biomarker is chosen from HHIP, PDLIM3, and SFRP1. In some embodiments, the brain tumor biomarker is ATOH1. In some embodiments, the brain tumor biomarker is EYA1. In some embodiments, the brain tumor biomarker is HHIP. In some embodiments, the brain tumor biomarker is PDLIM3. In some embodiments, the brain tumor biomarker is SFRP1. In some embodiments, the brain tumor is an shh subgroup brain tumor.

In some embodiments, the brain tumor biomarker is chosen from Natriuretic Peptide Receptor-3 (NPR3), Interphotoreceptor Matrix Proteoglycan 2 (IMPG2), Gamma-Aminobutyric Acid Type A Receptor Alpha 5 Subunit (GABRA5), EGF-Like-Domain, Multiple 11 (EGFL11), Mab-21 Like 2 (MAB21L2), and Myc. In some embodiments, the brain tumor biomarker is chosen from NPR3, IMPG2, GABRA5, EGFL11, and MAB21L2. In some embodiments, the brain tumor biomarker is NPR3. In some embodiments, the brain tumor biomarker is IMPG2. In some embodiments, the brain tumor biomarker is GABRA5. In some embodiments, the brain tumor biomarker is EGFL11. In some embodiments, the brain tumor biomarker is MAB21L2. In some embodiments, the brain tumor biomarker is Myc. In some embodiments, the brain tumor is a Group 3 subgroup brain tumor.

In some embodiments, the brain tumor biomarker is chosen from Potassium Voltage-Gated Channel Subfamily A Member 1 (KCNA1), Eomesodermin (EOMES), KH RNA Binding Domain Containing, Signal Transduction Associated 2 (KHDRBS2), RNA Binding Motif Protein 24 (RBM24), Unc-5 Netrin Receptor D (UNC5D), and 2′-5′-Oligoadenylate Synthetase 1 (OAS1). In some embodiments, the brain tumor biomarker is KCNA1. In some embodiments, the brain tumor biomarker is EOMES. In some embodiments, the brain tumor biomarker is KHDRBS2. In some embodiments, the brain tumor biomarker is RBM24. In some embodiments, the brain tumor biomarker is UNC5D. In some embodiments, the brain tumor biomarker is OAS1. In some embodiments, the brain tumor is a Group 4 subgroup brain tumor.

In some embodiments, the exosomal sample is a bodily fluid sample. In some embodiments, the bodily fluid is peripheral blood, sera, plasma, or cerebrospinal fluid (CSF). In some embodiments, the bodily fluid is peripheral blood. In some embodiments, the bodily fluid is sera. In some embodiments, the bodily fluid is plasma. In some embodiments, the bodily fluid is CSF. In some embodiments, the exosomal sample comprises plasma exosomes. In some embodiments, the exosomal sample comprises CSF exosomes.

In some embodiments, the one or more brain tumor biomarkers are mRNA biomarkers, protein biomarkers, or miRNA biomarkers. In some embodiments, the one or more brain tumor biomarkers are mRNA biomarkers. In some embodiments, the one or more brain tumor biomarkers are protein biomarkers. In some embodiments, the one or more brain tumor biomarkers are miRNA biomarkers.

Suitable methods for measuring protein expression levels in exosomes include, for example, contacting the exosomal sample with one or more detectable reagents that are suitable for measuring protein expression including, but not limited to, a labeled antibody or a primary antibody used in conjunction with a secondary antibody, and measuring protein expression levels based on the level of detectable reagent such as, for example, a fluorescent moiety or dye, in the exosomal sample after normalizing to total protein in the sample. Suitable methods for detecting protein expression level in an exosome sample include, but are not limited to, Western blot, immunoprecipitation, enzyme-linked immunosorbent assay (ELISA), radioimmunoassay (RIA), or fluorescent activated cell sorting (FACS). The measured protein expression level in the exosomal sample is compared to the protein expression level measured in a reference exosomal sample and the type of brain tumor is identified based on this comparison.

Suitable methods for measuring mRNA biomarker expression levels in exosomes include, for example, contacting the exosomal sample with one or more detectable reagents that are suitable for measuring mRNA expression including, but not limited to, an oligonucleotide that is complementary to the target biomarker mRNA comprising a label such as, for example, a fluorescent moiety or dye, in the exosomal sample after normalizing to total mRNA in the exosomal sample. Suitable methods for measuring mRNA expression levels also include, but are not limited to, Southern blot analysis, Northern blot analysis, and microarrays. The measured biomarker mRNA levels in the exosomal sample are compared to the biomarker mRNA levels measured in a reference exosomal sample and the type of brain tumor is identified based on this comparison.

In some embodiments, measuring the level of one or more brain tumor biomarker mRNA involves amplifying at least a portion of one or more nucleic acid molecules that encode brain tumor mRNA biomarkers, labeling the amplified nucleic acid molecule with a detectable label, contacting the labeled nucleic acid molecule with a support comprising one or more specific probes which hybridizes under stringent conditions to the nucleic acid sequence of the one or more amplified nucleic acid molecules, and detecting the detectable label.

In some embodiments, the levels of the corresponding one or more brain tumor biomarkers in a reference exosomal sample comprise the average exosomal brain tumor biomarker level in one or more exosomal samples from healthy, cancer-free humans. In some embodiments, the levels of the corresponding one or more brain tumor biomarkers in a reference exosomal sample comprise the brain tumor biomarker expression level in one or more exosomal samples from the human obtained at an earlier timepoint. A human having an elevated exosome level of one or more brain tumor biomarkers compared to the reference exosome levels is at risk of developing a brain tumor or already has a brain tumor and is a suitable candidate for treatment with an anti-exosome therapeutic agent.

In some embodiments, the anti-exosome therapeutic agent is an inhibitor of neutral sphingomyelinase and exosome biogenesis. In some embodiments, the anti-exosome therapeutic agent is GW4869 (N,N′-Bis[4-(4,5-dihydro-1H-imidazol-2-yl)phenyl]-3,3′-p-phenylene-bis-acrylamide dihydrochloride).

In some embodiments, the anti-exosome therapeutic agent is an inhibitor of the secretion of exosomes. In some embodiments, the anti-exosome therapeutic agent is dimethyl amiloride (DMA), neticonazole, ketoconazole, tipifarnib, isoproterenol, climbazole, triadimenol, Manumycin A, sulfisoxazole, or cannabidiol, or any combination thereof. In some embodiments, the anti-exosome therapeutic agent is DMA. In some embodiments, the anti-exosome therapeutic agent is neticonazole. In some embodiments, the anti-exosome therapeutic agent is ketoconazole. In some embodiments, the anti-exosome therapeutic agent is tipifarnib. In some embodiments, the anti-exosome therapeutic agent is isoproterenol. In some embodiments, the anti-exosome therapeutic agent is climbazole. In some embodiments, the anti-exosome therapeutic agent is triadimenol. In some embodiments, the anti-exosome therapeutic agent is Manumycin A. In some embodiments, the anti-exosome therapeutic agent is sulfisoxazole. In some embodiments, the anti-exosome therapeutic agent is cannabidiol.

In some embodiments, the anti-exosome therapeutic agent is a Ras-related (Rab) protein inhibitor. In some embodiments, the Rab inhibitor is a Rab27a inhibitor, a Rab5b inhibitor, a Rab7 inhibitor, a Rab la inhibitor, or any combination thereof. In some embodiments, the anti-exosome therapeutic agent is an inhibitory nucleic acid molecule including, but not limited to, antisense molecules, siRNA molecules, shRNA molecules, and microRNA molecules. In some embodiments, the anti-exosome therapeutic agent is an inhibitor of microtubules movement (such as taxol), an Hsp90/Hsp70 inhibitor, a golgi-ER transport inhibitor (such as brefeldin), and an mTOR inhibitor.

In some embodiments, the methods further comprise administering to the human one or more additional therapeutic agents. In some embodiments, the one or more additional therapeutic agents are chosen from a chemotherapeutic agent, a radiotherapeutic agent, an anti-angiogenic agent, a premetastatic niche formation inhibitor, and a stromal inhibitor or any combination thereof. In some embodiments, the one or more additional therapeutic agent is a chemotherapeutic agent. In some embodiments, the one or more additional therapeutic agent is a radiotherapeutic agent. In some embodiments, the one or more additional therapeutic agent is an anti-angiogenic agent. In some embodiments, the one or more additional therapeutic agent is a premetastatic niche formation inhibitor. In some embodiments, the one or more additional therapeutic agent is a stromal inhibitor.

In some embodiments, the additional therapeutic agent is chosen from carmustine, temozolomide, bevacizumab, larotrectinib, everolimus, vincristine, lomustine, procarbazine, vismodegib, sonidegib, erlotinib, and glasdegib, or any combination thereof. In some embodiments, the therapeutic agent is chosen from vismodegib, sonidegib, and glasdegib. In some embodiments, the additional therapeutic agent is a combination of procarbazine, lomustine, and vincristine. In some embodiments, the additional therapeutic agent is carmustine. In some embodiments, the additional therapeutic agent is temozolomide. In some embodiments, the additional therapeutic agent is bevacizumab. In some embodiments, the additional therapeutic agent is larotrectinib. In some embodiments, the additional therapeutic agent is everolimus. In some embodiments, the additional therapeutic agent is vincristine. In some embodiments, the additional therapeutic agent is lomustine. In some embodiments, the additional therapeutic agent is procarbazine. In some embodiments, the additional therapeutic agent is vismodegib. In some embodiments, the additional therapeutic agent is sonidegib. In some embodiments, the additional therapeutic agent is glasdegib. In some embodiments, the additional therapeutic agent is erlotinib.

In some embodiments, the additional therapeutic agent is chosen from BiCNU® (carmustine), TEMODAR® (temozolomide), AVASTIN® or MVASI® (bevacizumab), VITRAKVI® (larotrectinib), AFINITOR® (everolimus), ONCOVIN® or VINCASAR® (vincristine), GLEOSTINE® (lomustine), MATULANE® (procarbazine), Erivedge® (vismodegib), ODOMZO® (sonidegib), TARCEVA® (erlotinib), and VENCLEXTA® (glasdegib), or any combination thereof. In some embodiments, the therapeutic agent is chosen from ERIVEDGE® (vismodegib), ODOMZO® (sonidegib), and VENCLEXTA® (glasdegib). In some embodiments, the additional therapeutic agent is a combination of MATULANE® (procarbazine), GLEOSTINE® (lomustine), and ONCOVIN® or VINCASAR® (vincristine). In some embodiments, the additional therapeutic agent is BiCNU® (carmustine). In some embodiments, the additional therapeutic agent is TEMODAR® (temozolomide). In some embodiments, the additional therapeutic agent is AVASTIN® or MVASI® (bevacizumab). In some embodiments, the additional therapeutic agent is VITRAKVI® (larotrectinib). In some embodiments, the additional therapeutic agent is AFINITOR® (everolimus). In some embodiments, the additional therapeutic agent is ONCOVIN® or VINCASAR® (vincristine). In some embodiments, the additional therapeutic agent is GLEOSTINE® (lomustine). In some embodiments, the additional therapeutic agent is MATULANE® (procarbazine). In some embodiments, the additional therapeutic agent is ERIVEDGE® (vismodegib). In some embodiments, the additional therapeutic agent is ODOMZO® (sonidegib). In some embodiments, the additional therapeutic agent is VENCLEXTA® (glasdegib). In some embodiments, the additional therapeutic agent is TARCEVA® (erlotinib).

In some embodiments, the human is administered a combination of GW4869 or DMA with any one of vismodegib, cisplatin, and temozolomide. In some embodiments, the human is administered a combination of GW4869 and vismodegib, GW4869 and cisplatin, or GW4869 and temozolomide. In some embodiments, the human is administered a combination of GW4869 and vismodegib. In some embodiments, the human is administered a combination of GW4869 and cisplatin. In some embodiments, the human is administered a combination of GW4869 and temozolomide.

Assaying the level of one or more brain tumor biomarkers in exosomes can be carried out by any of the methods described herein. In some embodiments, these methods can be carried out in vitro. In some embodiments, these methods can be carried out in situ. In some embodiments, these methods can be carried out in vivo. In any of these embodiments, the brain tumor biomarkers can be present within exosomes obtained from the human subject.

Methods for isolating and purifying exosomes from a blood or plasma sample for measuring brain tumor biomarker levels are described in, for example, PCT Publication Nos. WO 2012/006476, WO 2013/071239, WO 2013/158203, WO 2018/112557, WO 2014/159662, WO 2015/131153, and WO 2016/172598. In some embodiments, the exosome is isolated from the exosomal sample by size exclusion chromatography, density gradient centrifugation, differential centrifugation, nanomembrane ultrafiltration, immunoabsorbent capture, affinity purification, microfluidic separation, polymer-based precipitation, or any combination thereof. In some embodiments, the exosome is isolated from the exosomal sample by size exclusion chromatography. In some embodiments, the exosome is isolated from the exosomal sample by density gradient centrifugation. In some embodiments, the exosome is isolated from the exosomal sample by differential centrifugation. In some embodiments, the exosome is isolated from the exosomal sample by nanomembrane ultrafiltration. In some embodiments, the exosome is isolated from the exosomal sample by immunoabsorbent capture. In some embodiments, the exosome is isolated from the exosomal sample by affinity purification. In some embodiments, the exosome is isolated from the exosomal sample by microfluidic separation. In some embodiments, the exosome is isolated from the exosomal sample by polymer-based precipitation.

The present disclosure also provides methods of classifying a medulloblastoma tumor in a human. The methods comprise assaying the level of one or more medulloblastoma biomarkers chosen from CTTNB1, DKK1, WIF1, TNC, GAD1, DDK2, EMX2, ATOH1, EYA1, HHIP, PDLIM3, SFRP1, NPR3, IMPG2, GABRA5, EGFL11, MAB21L2, KCNA1, EOMES, KHDRBS2, RBM24, UNC5D, and OAS1, in an exosomal sample obtained from the human. The methods also comprise comparing the level of the one or more biomarkers in the exosomal sample from the human to the levels of the corresponding one or more biomarkers in a reference exosomal sample. An increase in the level of one or more of ATOH1, EYA1, HHIP, PDLIM3, and SFRP1 in the exosomal sample from the human compared to the reference exosomal sample indicates the presence of a sonic hedgehog subgroup medulloblastoma. An increase in the level of one or more of CTTNB1, DKK1, WIF1, TNC, GAD1, DDK2, and EMX2 in the exosomal sample from the human compared to the reference exosomal sample indicates the presence of a Wnt subgroup medulloblastoma. An increase in the level of one or more of NPR3, IMPG2, GABRA5, EGFL11, and MAB21L2 in the exosomal sample from the human compared to the reference exosomal sample indicates the presence of a Group C medulloblastoma. An increase in the level of one or more of KCNA1, EOMES, KHDRBS2, RBM24, UNC5D, and OAS1 in the exosomal sample from the human compared to the reference exosomal sample indicates the presence of a Group D medulloblastoma. In some embodiments, the methods also comprise administering an anti-exosome therapeutic agent to the human.

In these methods, the medulloblastoma can be any of the medulloblastoma subgroups described herein. In these methods, the medulloblastoma biomarkers can be any of the biomarkers, or combinations thereof, described herein (i.e., any of the Wnt subgroups biomarkers, shh subgroup biomarkers, Group 3 subgroup biomarkers, or Group 4 subgroup biomarkers).

In these methods, the exosomal sample can be any of the bodily fluid samples described herein, and can be any of the exosomal samples described herein.

In these methods, the one or more medulloblastoma biomarkers can be mRNA biomarkers, protein biomarkers, and/or miRNA biomarkers.

In these methods, the levels of the corresponding one or more medulloblastoma biomarkers in a reference exosomal sample can comprise the average exosomal medulloblastoma biomarker level in one or more samples from healthy, cancer-free humans. In these methods, the levels of the corresponding one or more medulloblastoma biomarkers in a reference exosomal sample can comprise the exosomal medulloblastoma biomarker expression level in one or more samples from the human obtained at an earlier timepoint.

In these methods, the exosome is isolated from the exosomal sample by any of the methods described herein.

In these methods, the anti-exosome therapeutic agent is any of the anti-exosome therapeutic agents described herein.

In any of these methods, the human can be further administering any one or more of any of the additional therapeutic agents described herein.

The present disclosure also provides methods of treating a human having a brain tumor comprising administering to the human in need thereof an anti-exosome therapeutic agent. In some embodiments, the methods inhibit or slow brain tumor progression.

In some embodiments, the brain tumor is a glioblastoma. In some embodiments, the brain tumor is a medulloblastoma.

In some embodiments, the brain tumor is a Wingless (Wnt) subgroup brain tumor, a sonic hedgehog (shh) subgroup brain tumor, a Group 3 subgroup brain tumor, or a Group 4 subgroup brain tumor. In some embodiments, the brain tumor is a Wnt subgroup brain tumor. In some embodiments, the brain tumor is an shh subgroup brain tumor. In some embodiments, the brain tumor is a Group 3 subgroup brain tumor. In some embodiments, the brain tumor is a Group 4 subgroup brain tumor.

In some embodiments, the anti-exosome therapeutic agent is an inhibitor of neutral sphingomyelinase and exosome biogenesis. In some embodiments, the anti-exosome therapeutic agent is GW4869 (N,N′-Bis[4-(4,5-dihydro-1H-imidazol-2-yl)phenyl]-3,3′-p-phenylene-bis-acrylamide dihydrochloride).

In some embodiments, the anti-exosome therapeutic agent is an inhibitor of the secretion of exosomes. In some embodiments, the anti-exosome therapeutic agent is dimethyl amiloride (DMA), neticonazole, ketoconazole, tipifarnib, isoproterenol, climbazole, triadimenol, Manumycin A, sulfisoxazole, or cannabidiol, or any combination thereof.

In some embodiments, the anti-exosome therapeutic agent is a Ras-related (Rab) protein inhibitor. In some embodiments, the Rab inhibitor is a Rab27a inhibitor, a Rab5b inhibitor, a Rab7 inhibitor, a Rab la inhibitor, or any combination thereof. In some embodiments, the anti-exosome therapeutic agent is an inhibitory nucleic acid molecule including, but not limited to, antisense molecules, siRNA molecules, shRNA molecules, and microRNA molecules. In some embodiments, the anti-exosome therapeutic agent is an inhibitor of microtubules movement (such as taxol), an Hsp90/Hsp70 inhibitor, a golgi-ER transport inhibitor (such as brefeldin), and an mTOR inhibitor.

In some embodiments, the methods further comprise administering to the human one or more additional therapeutic agents. In some embodiments, the one or more additional therapeutic agents are chosen from a chemotherapeutic agent, a radiotherapeutic agent, an anti-angiogenic agent, a premetastatic niche formation inhibitor, and a stromal inhibitor or any combination thereof. In some embodiments, the one or more additional therapeutic agent is a chemotherapeutic agent. In some embodiments, the one or more additional therapeutic agent is a radiotherapeutic agent. In some embodiments, the one or more additional therapeutic agent is an anti-angiogenic agent. In some embodiments, the one or more additional therapeutic agent is a premetastatic niche formation inhibitor. In some embodiments, the one or more additional therapeutic agent is a stromal inhibitor.

In some embodiments, the additional therapeutic agent is chosen from carmustine, temozolomide, bevacizumab, larotrectinib, everolimus, vincristine, lomustine, procarbazine, vismodegib, sonidegib, erlotinib, and glasdegib, or any combination thereof. In some embodiments, the therapeutic agent is chosen from vismodegib, sonidegib, and glasdegib. In some embodiments, the additional therapeutic agent is a combination of procarbazine, lomustine, and vincristine. In some embodiments, the additional therapeutic agent is carmustine. In some embodiments, the additional therapeutic agent is temozolomide. In some embodiments, the additional therapeutic agent is bevacizumab. In some embodiments, the additional therapeutic agent is larotrectinib. In some embodiments, the additional therapeutic agent is everolimus. In some embodiments, the additional therapeutic agent is vincristine. In some embodiments, the additional therapeutic agent is lomustine. In some embodiments, the additional therapeutic agent is procarbazine. In some embodiments, the additional therapeutic agent is vismodegib. In some embodiments, the additional therapeutic agent is sonidegib. In some embodiments, the additional therapeutic agent is glasdegib. In some embodiments, the additional therapeutic agent is erlotinib.

In some embodiments, the additional therapeutic agent is chosen from BiCNU® (carmustine), TEMODAR® (temozolomide), AVASTIN® or MVASI® (bevacizumab), VITRAKVI® (larotrectinib), AFINITOR® (everolimus), ONCOVIN® or VINCASAR® (vincristine), GLEOSTINE® (lomustine), MATULANE® (procarbazine), Erivedge® (vismodegib), ODOMZO® (sonidegib), TARCEVA® (erlotinib), and VENCLEXTA® (glasdegib), or any combination thereof. In some embodiments, the therapeutic agent is chosen from ERIVEDGE® (vismodegib), ODOMZO® (sonidegib), and VENCLEXTA® (glasdegib). In some embodiments, the additional therapeutic agent is a combination of MATULANE® (procarbazine), GLEOSTINE® (lomustine), and ONCOVIN® or VINCASAR® (vincristine). In some embodiments, the additional therapeutic agent is BiCNU® (carmustine). In some embodiments, the additional therapeutic agent is TEMODAR® (temozolomide). In some embodiments, the additional therapeutic agent is AVASTIN® or MVASI® (bevacizumab). In some embodiments, the additional therapeutic agent is VITRAKVI® (larotrectinib). In some embodiments, the additional therapeutic agent is AFINITOR® (everolimus). In some embodiments, the additional therapeutic agent is ONCOVIN® or VINCASAR® (vincristine). In some embodiments, the additional therapeutic agent is GLEOSTINE® (lomustine). In some embodiments, the additional therapeutic agent is MATULANE® (procarbazine). In some embodiments, the additional therapeutic agent is ERIVEDGE® (vismodegib). In some embodiments, the additional therapeutic agent is ODOMZO® (sonidegib). In some embodiments, the additional therapeutic agent is VENCLEXTA® (glasdegib). In some embodiments, the additional therapeutic agent is TARCEVA® (erlotinib).

In some embodiments, the human is administered a combination of GW4869 or DMA with any one of vismodegib, cisplatin, and temozolomide. In some embodiments, the human is administered a combination of GW4869 and vismodegib, GW4869 and cisplatin, or GW4869 and temozolomide. In some embodiments, the human is administered a combination of GW4869 and vismodegib. In some embodiments, the human is administered a combination of GW4869 and cisplatin. In some embodiments, the human is administered a combination of GW4869 and temozolomide.

In some embodiments, the human is determined to have the brain tumor by any of the methods described herein.

The treatment methods described herein can be performed concurrently or consecutively with other therapeutic approaches including, but not limited to, combination therapy, chemotherapy, immunotherapy, radiation therapy (such as, external beam radiation therapy or brachytherapy), anti-angiogenic therapy, adjuvant therapy, surgery, and bone-marrow therapy.

The administration of any of the agents described herein can be carried out systemically or via direct or local administration to the tumor site. Suitable modes of systemic administration include, but are not limited to, orally, topically, transdermally, parenterally, intradermally, intramuscularly, intraperitoneally, intravenously, subcutaneously, or by intranasal instillation, by intracavitary or intravesical instillation, intraocularly, intraarterially, intralesionally, or by application to mucous membranes. Suitable modes of local administration include, but are not limited to, catheterization, implantation, direct injection, dermal/transdermal application, intrathecally, and portal vein administration to relevant tissues, or by any other local administration technique.

The present disclosure also provides methods of suppressing vismodegib resistance in a human having a vismodegib-resistant brain tumor. The methods comprise administering to the human in need thereof an anti-exosome therapeutic agent.

In some embodiments, the brain tumor is a glioblastoma. In some embodiments, the brain tumor is a medulloblastoma.

In some embodiments, the brain tumor is a Wingless (Wnt) subgroup brain tumor, a sonic hedgehog (shh) subgroup brain tumor, a Group 3 subgroup brain tumor, or a Group 4 subgroup brain tumor. In some embodiments, the brain tumor is a Wnt subgroup brain tumor. In some embodiments, the brain tumor is an shh subgroup brain tumor. In some embodiments, the brain tumor is a Group 3 subgroup brain tumor. In some embodiments, the brain tumor is a Group 4 subgroup brain tumor.

In some embodiments, the anti-exosome therapeutic agent is an inhibitor of neutral sphingomyelinase and exosome biogenesis. In some embodiments, the anti-exosome therapeutic agent is GW4869 (N,N′-Bis[4-(4,5-dihydro-1H-imidazol-2-yl)phenyl]-3,3′-p-phenylene-bis-acrylamide dihydrochloride).

In some embodiments, the anti-exosome therapeutic agent is an inhibitor of the secretion of exosomes. In some embodiments, the anti-exosome therapeutic agent is dimethyl amiloride (DMA), neticonazole, ketoconazole, tipifarnib, isoproterenol, climbazole, triadimenol, Manumycin A, sulfisoxazole, or cannabidiol, or any combination thereof.

In some embodiments, the anti-exosome therapeutic agent is a Ras-related (Rab) protein inhibitor. In some embodiments, the Rab inhibitor is a Rab27a inhibitor, a Rab5b inhibitor, a Rab7 inhibitor, a Rab la inhibitor, or any combination thereof. In some embodiments, the anti-exosome therapeutic agent is an inhibitory nucleic acid molecule including, but not limited to, antisense molecules, siRNA molecules, shRNA molecules, and microRNA molecules. In some embodiments, the anti-exosome therapeutic agent is an inhibitor of microtubules movement (such as taxol), an Hsp90/Hsp70 inhibitor, a golgi-ER transport inhibitor (such as brefeldin), and an mTOR inhibitor.

In some embodiments, the methods further comprise administering to the human one or more additional therapeutic agents. In some embodiments, the one or more additional therapeutic agents are chosen from a chemotherapeutic agent, a radiotherapeutic agent, an anti-angiogenic agent, a premetastatic niche formation inhibitor, and a stromal inhibitor or any combination thereof. In some embodiments, the one or more additional therapeutic agent is a chemotherapeutic agent. In some embodiments, the one or more additional therapeutic agent is a radiotherapeutic agent. In some embodiments, the one or more additional therapeutic agent is an anti-angiogenic agent. In some embodiments, the one or more additional therapeutic agent is a premetastatic niche formation inhibitor. In some embodiments, the one or more additional therapeutic agent is a stromal inhibitor.

In some embodiments, the additional therapeutic agent is chosen from carmustine, temozolomide, bevacizumab, larotrectinib, everolimus, vincristine, lomustine, procarbazine, vismodegib, sonidegib, erlotinib, and glasdegib, or any combination thereof. In some embodiments, the therapeutic agent is chosen from vismodegib, sonidegib, and glasdegib. In some embodiments, the additional therapeutic agent is a combination of procarbazine, lomustine, and vincristine. In some embodiments, the additional therapeutic agent is carmustine. In some embodiments, the additional therapeutic agent is temozolomide. In some embodiments, the additional therapeutic agent is bevacizumab. In some embodiments, the additional therapeutic agent is larotrectinib. In some embodiments, the additional therapeutic agent is everolimus. In some embodiments, the additional therapeutic agent is vincristine. In some embodiments, the additional therapeutic agent is lomustine. In some embodiments, the additional therapeutic agent is procarbazine. In some embodiments, the additional therapeutic agent is vismodegib. In some embodiments, the additional therapeutic agent is sonidegib. In some embodiments, the additional therapeutic agent is glasdegib. In some embodiments, the additional therapeutic agent is erlotinib.

In some embodiments, the additional therapeutic agent is chosen from BiCNU® (carmustine), TEMODAR® (temozolomide), AVASTIN® or MVASI® (bevacizumab), VITRAKVI® (larotrectinib), AFINITOR® (everolimus), ONCOVIN® or VINCASAR® (vincristine), GLEOSTINE® (lomustine), MATULANE® (procarbazine), Erivedge® (vismodegib), ODOMZO® (sonidegib), TARCEVA® (erlotinib), and VENCLEXTA® (glasdegib), or any combination thereof. In some embodiments, the therapeutic agent is chosen from ERIVEDGE® (vismodegib), ODOMZO® (sonidegib), and VENCLEXTA® (glasdegib). In some embodiments, the additional therapeutic agent is a combination of MATULANE® (procarbazine), GLEOSTINE® (lomustine), and ONCOVIN® or VINCASAR® (vincristine). In some embodiments, the additional therapeutic agent is BiCNU® (carmustine). In some embodiments, the additional therapeutic agent is TEMODAR® (temozolomide). In some embodiments, the additional therapeutic agent is AVASTIN® or MVASI® (bevacizumab). In some embodiments, the additional therapeutic agent is VITRAKVI® (larotrectinib). In some embodiments, the additional therapeutic agent is AFINITOR® (everolimus). In some embodiments, the additional therapeutic agent is ONCOVIN® or VINCASAR® (vincristine). In some embodiments, the additional therapeutic agent is GLEOSTINE® (lomustine). In some embodiments, the additional therapeutic agent is MATULANE® (procarbazine). In some embodiments, the additional therapeutic agent is ERIVEDGE® (vismodegib). In some embodiments, the additional therapeutic agent is ODOMZO® (sonidegib). In some embodiments, the additional therapeutic agent is VENCLEXTA® (glasdegib). In some embodiments, the additional therapeutic agent is TARCEVA® (erlotinib).

In some embodiments, the human is administered a combination of GW4869 or DMA with any one of vismodegib, cisplatin, and temozolomide. In some embodiments, the human is administered a combination of GW4869 and vismodegib, GW4869 and cisplatin, or GW4869 and temozolomide. In some embodiments, the human is administered a combination of GW4869 and vismodegib. In some embodiments, the human is administered a combination of GW4869 and cisplatin. In some embodiments, the human is administered a combination of GW4869 and temozolomide.

The present disclosure also provides methods of monitoring brain tumor treatment in a human. The methods comprise assaying the level of one or more brain tumor biomarkers in a first exosomal sample obtained from the human and a second exosomal sample obtained from the human, wherein the second exosomal sample is obtained from the human after the first exosomal sample. The methods also comprise comparing the level of the one or more brain tumor biomarkers in the first exosomal sample to the level of the one or more brain tumor biomarkers in the second exosomal sample. A decrease in the level of the one or more brain tumor biomarkers in the second exosomal sample compared to the first exosomal sample indicates the human is responding favorably to the brain tumor treatment. No change or an increase in the level of the one or more brain tumor biomarkers in the second exosomal sample compared to the first exosomal sample indicates the human is not responding favorably to the brain tumor treatment.

In some embodiments, the first exosomal sample is obtained from the human prior to initiation of treatment and the second exosomal sample is obtained from the human after initiation of treatment. In some embodiments, the first exosomal sample is obtained from the human after the human is diagnosed with the brain tumor and before the initiation of treatment, and the second exosomal sample is obtained from the human within one month after the initiation of treatment.

In some embodiments, the brain tumor is a glioblastoma. In some embodiments, the brain tumor is a medulloblastoma.

In some embodiments, the brain tumor is a glioblastoma. In some embodiments, the brain tumor is a medulloblastoma.

In some embodiments, the brain tumor is a Wingless (Wnt) subgroup brain tumor, a sonic hedgehog (shh) subgroup brain tumor, a Group 3 subgroup brain tumor, or a Group 4 subgroup brain tumor. In some embodiments, the brain tumor is a Wnt subgroup brain tumor. In some embodiments, the brain tumor is an shh subgroup brain tumor. In some embodiments, the brain tumor is a Group 3 subgroup brain tumor. In some embodiments, the brain tumor is a Group 4 subgroup brain tumor.

In some embodiments, the brain tumor biomarker is chosen from Catenin Beta 1 (CTTNB1), Dickkopf WNT Signaling Pathway Inhibitor 1 (DKK1), Wnt Inhibitory Factor 1 (WIF1), Tenascin C (TNC), Glutamate Decarboxylase 1 (GAD1), Dickkopf WNT Signaling Pathway Inhibitor 2 (DDK2), and Empty Spiracles Homeobox 2 (EMX2), or any combination thereof. In some embodiments, the brain tumor biomarker is chosen from WIF1, TNC, GAD1, DDK2, and EMX2, or any combination thereof. In some embodiments, the brain tumor biomarker is CTTNB1. In some embodiments, the brain tumor biomarker is DKK1. In some embodiments, the brain tumor biomarker is WIF1. In some embodiments, the brain tumor biomarker is TNC. In some embodiments, the brain tumor biomarker is GAD1. In some embodiments, the brain tumor biomarker is DDK2. In some embodiments, the brain tumor biomarker is EMX2. In some embodiments, the brain tumor is a Wnt subgroup brain tumor.

In some embodiments, the brain tumor biomarker is chosen from Atonal BHLH Transcription Factor 1 (ATOH1), EYA Transcriptional Coactivator and Phosphatase 1 (EYA1), Hedgehog-Interacting Protein (HHIP), PDZ and LIM Domain Protein 3 (PDLIM3), and Secreted Frizzled-Related Protein 1 (SFRP1). In some embodiments, the brain tumor biomarker is chosen from HHIP, PDLIM3, and SFRP1. In some embodiments, the brain tumor biomarker is ATOH1. In some embodiments, the brain tumor biomarker is EYA1. In some embodiments, the brain tumor biomarker is HHIP. In some embodiments, the brain tumor biomarker is PDLIM3. In some embodiments, the brain tumor biomarker is SFRP1. In some embodiments, the brain tumor is an shh subgroup brain tumor.

In some embodiments, the brain tumor biomarker is chosen from Natriuretic Peptide Receptor-3 (NPR3), Interphotoreceptor Matrix Proteoglycan 2 (IMPG2), Gamma-Aminobutyric Acid Type A Receptor Alpha 5 Subunit (GABRA5), EGF-Like-Domain, Multiple 11 (EGFL11), Mab-21 Like 2 (MAB21L2), and Myc. In some embodiments, the brain tumor biomarker is chosen from NPR3, IMPG2, GABRA5, EGFL11, and MAB21L2. In some embodiments, the brain tumor biomarker is NPR3. In some embodiments, the brain tumor biomarker is IMPG2. In some embodiments, the brain tumor biomarker is GABRA5. In some embodiments, the brain tumor biomarker is EGFL11. In some embodiments, the brain tumor biomarker is MAB21L2. In some embodiments, the brain tumor biomarker is Myc. In some embodiments, the brain tumor is a Group 3 subgroup brain tumor.

In some embodiments, the brain tumor biomarker is chosen from Potassium Voltage-Gated Channel Subfamily A Member 1 (KCNA1), Eomesodermin (EOMES), KH RNA Binding Domain Containing, Signal Transduction Associated 2 (KHDRBS2), RNA Binding Motif Protein 24 (RBM24), Unc-5 Netrin Receptor D (UNC5D), and 2′-5′-Oligoadenylate Synthetase 1 (OAS1). In some embodiments, the brain tumor biomarker is KCNA1. In some embodiments, the brain tumor biomarker is EOMES. In some embodiments, the brain tumor biomarker is KHDRBS2. In some embodiments, the brain tumor biomarker is RBM24. In some embodiments, the brain tumor biomarker is UNC5D. In some embodiments, the brain tumor biomarker is OAS1. In some embodiments, the brain tumor is a Group 4 subgroup brain tumor.

In some embodiments, the exosomal sample is a bodily fluid sample. In some embodiments, the bodily fluid is peripheral blood, sera, plasma, or cerebrospinal fluid (CSF). In some embodiments, the bodily fluid is peripheral blood. In some embodiments, the bodily fluid is sera. In some embodiments, the bodily fluid is plasma. In some embodiments, the bodily fluid is CSF. In some embodiments, the exosomal sample comprises plasma exosomes. In some embodiments, the exosomal sample comprises CSF exosomes.

In some embodiments, the one or more brain tumor biomarkers are mRNA biomarkers, protein biomarkers, or miRNA biomarkers. In some embodiments, the one or more brain tumor biomarkers are mRNA biomarkers. In some embodiments, the one or more brain tumor biomarkers are protein biomarkers. In some embodiments, the one or more brain tumor biomarkers are miRNA biomarkers.

In some embodiments, the brain tumor treatment is chemotherapy, immunotherapy, radiotherapy, anti-angiogenic therapy, or surgery.

In some embodiments, the methods further comprises modifying the course of treatment for the human. In some embodiments, when there is no change or there is an increase in the level of the one or more brain tumor biomarkers in the second exosomal sample compared to the first exosomal sample, the modification of treatment comprises administering an anti-exosome therapeutic agent to the human.

In some embodiments, the anti-exosome therapeutic agent is an inhibitor of neutral sphingomyelinase and exosome biogenesis. In some embodiments, the anti-exosome therapeutic agent is GW4869 (N,N′-Bis[4-(4,5-dihydro-1H-imidazol-2-yl)phenyl]-3,3′-p-phenylene-bis-acrylamide dihydrochloride).

In some embodiments, the anti-exosome therapeutic agent is an inhibitor of the secretion of exosomes. In some embodiments, the anti-exosome therapeutic agent is dimethyl amiloride (DMA), neticonazole, ketoconazole, tipifarnib, isoproterenol, climbazole, triadimenol, Manumycin A, sulfisoxazole, or cannabidiol, or any combination thereof.

In some embodiments, the anti-exosome therapeutic agent is a Ras-related (Rab) protein inhibitor. In some embodiments, the Rab inhibitor is a Rab27a inhibitor, a Rab5b inhibitor, a Rab7 inhibitor, a Rab la inhibitor, or any combination thereof. In some embodiments, the anti-exosome therapeutic agent is an inhibitory nucleic acid molecule including, but not limited to, antisense molecules, siRNA molecules, shRNA molecules, and microRNA molecules. In some embodiments, the anti-exosome therapeutic agent is an inhibitor of microtubules movement (such as taxol), an Hsp90/Hsp70 inhibitor, a golgi-ER transport inhibitor (such as brefeldin), and an mTOR inhibitor.

In some embodiments, the human is administered a combination of GW4869 or DMA with any one of vismodegib, cisplatin, and temozolomide. In some embodiments, the human is administered a combination of GW4869 and vismodegib, GW4869 and cisplatin, or GW4869 and temozolomide. In some embodiments, the human is administered a combination of GW4869 and vismodegib. In some embodiments, the human is administered a combination of GW4869 and cisplatin. In some embodiments, the human is administered a combination of GW4869 and temozolomide.

Additional treatment modifications in response to an unfavorable monitoring result include, but not limited to, substitution of one or more therapeutic agents, addition of one or more therapeutic agents to the treatment regimen, adjusting the therapeutic regimen (such as, dosage, administration frequency, route, or duration of treatment). For example, in some embodiments, the dose of the therapeutic agents can be increased by about 10%, by about 20%, by about 30%, by about 40%, by about 50%, by about 60%, by about 70%, by about 80%, or by about 90% for humans having an unfavorable monitoring result. In addition, the dose of therapeutic agents can be administered more frequently.

The present disclosure also provides methods of determining the prognosis of a human having a brain tumor. Prognosis generally refers to a determination of the likely outcome of an illness, in this case brain tumor. In some embodiments, the prognosis refers to a determination of the status or metastatic potential of a primary cancer or primary tumor. An unfavorable prognosis predicts the development or progression of brain tumor, whereas a favorable prognosis indicates brain tumor is not likely to develop or to progress.

The present disclosure also provides in vivo methods of identifying candidate compounds useful for inhibiting primary tumor growth or preventing the formation and progression of a brain tumor in a subject. In some embodiments, the methods involve providing a test compound and providing an animal model comprising a primary tumor. In some embodiments, the methods further comprise administering to the animal model malignant cell-derived exosomes and the test compound, and identifying test compounds which inhibit exosome activity in the animal model as candidate compounds useful for inhibiting primary tumor growth or preventing the formation and progression of a brain tumor in a human. Prior to and following administration of the test compound, samples from the animal (such as, a blood sample) can be analyzed for a change in the levels of one or more brain tumor biomarkers in exosomal samples. The endpoints can be analyzed in a number of ways such as, for example, measuring total exosome secretion, rate of secretion, total exosome protein, RNA, DNA content.

The present disclosure also provides in vitro methods of identifying candidate compounds useful for inhibiting primary tumor growth or preventing the formation and progression of a brain tumor in a subject. Suitable malignant cells for use in such method include, but are not limited to, CCF-STTG1, SW 1088, CHLA-02-ATRT, A172, U-138 MG, Hs 683, CHLA-01-MED, CHP-212, H4, D341 Med, PFSK-1, M059K, M059J, IMR-32, and T98G cells.

The present disclosure also provides anti-exosome therapeutic agents for use in treating a human having a brain tumor. The anti-exosome therapeutic agents can be any of the anti-exosome therapeutic agents described herein.

The present disclosure also provides anti-exosome therapeutic agents for use in the preparation of a medicament for treating a human having a brain tumor. The anti-exosome therapeutic agents can be any of the anti-exosome therapeutic agents described herein.

The present disclosure also provides anti-exosome therapeutic agents for use in suppressing vismodegib resistance in a human having a vismodegib-resistant brain tumor. The anti-exosome therapeutic agents can be any of the anti-exosome therapeutic agents described herein.

The present disclosure also provides anti-exosome therapeutic agents for use in the preparation of a medicament for suppressing vismodegib resistance in a human having a vismodegib-resistant brain tumor. The anti-exosome therapeutic agents can be any of the anti-exosome therapeutic agents described herein.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### Examples

### Example 1: Materials and Methods

### Exosome Isolation

Blood was collected from the Math1-Cre/Rosa-GFP/Ptch1^{-/-} mice in EDTA-Vacutainer blood collection tubes and centrifuged at 3000×g for 15 minutes to remove cells and cell debris to obtain plasma. The supernatant was filtered (under sterile conditions) through a 0.22 micron filter into a fresh conical tube. The filtered plasma was concentrated in Centrifugal Filter Unit in preparation for ultracentrifugation. Prior to adding plasma, the Centrifugal Filter Unit was primed through addition of 1-2 mL of PBS and centrifuged at 4.0×1000 rpm for 10-15 minutes or until most of the PBS had passed through the filter. The plasma was centrifuged at 4.0×1000 rpm for about 30-40 minutes or until the final volume of concentrated media was about 1.5 mL. The concentrated plasma was transferred to an ultracentrifuge tube, inserted into its rotor adaptor, and was adjusted using sterile PBS so that all samples/blanks were of equal weight to a hundredth of a gram. The samples were centrifuged in an Ultracentrifuge at 34.0×1000 rpm (100,000×G) for 1 hour. Subsequently, the supernatant was carefully removed. Because a swing bucket rotor was used, the exosome pellet appeared invisible, and was directly at the middle of the bottom of the tube. The exosome pellet was re-suspended and washed, and 1 mL of sterile PBS was added to re-suspend the pellet. The pellet was centrifuged again at 34.0×1000 rpm for 1 hour. The supernatant was again carefully removed, and the pellet was re-suspended in 40 μl of sterile PBS and transferred to a fresh microcentrifuge tube. The purified exosome samples (now ready for further analysis) were aliquoted into 10 μl samples and frozen at −80° C. for long term storage.

### Characterization of Exosome Number and Size

Measurement and analysis of exosomes was carried out by NanoSight ns300 Malvern (Worcester, UK) using approximately 1000 μl of exosomes preparations diluted in PBS (1000 times). Individual videos of 60 seconds for each sample were acquired using the maximum camera gain and analyzed by the NanoSight particle tracking software to determine particle density and size.

### Immunology

Cells were lysed and total protein concentration was measured by a bicinchoninic acid method. Thirty micrograms of protein were electrophoresed on a 10% SDS-PAGE gel and transferred to a polyvinylidene fluoride (PVDF) membrane (Millipore, Billerica, Mass., USA). The membrane was blocked and incubated with primary antibody (rabbit anti-GFP, 1:1000; rabbit anti-CD63, 1:1000) at 4° C. overnight. The membrane was washed with Tris-buffered saline containing 0.1% Tween-20 (TBST) and incubated with horseradish peroxidase-conjugated secondary antibody. The protein bands were detected by chemiluminescence (Sigma) and visualized using Biolmaging Systems.

### RNA Extraction

The exosome pellet was resuspended in 350 μl of Lysis Buffer, vortexed for 15 seconds, and placed at room temperature for 5 minutes to complete lysis. 200 μl of 100% ethanol was added to the resuspended exosomes and vortexed for 10 seconds. The sample was transferred to an ExoQuick RNA spin column and centrifuged at 13,000 rpm for 1 minute. The flow-through was discarded and the column was washed with 400 μl of Wash Buffer and centrifuged at 13,000 rpm for 1 minute. The flow-through was again discarded. The wash was repeated once more and the column was centrifuged at 13,000 rpm for 2 minutes to dry. The spin column was placed into a new, RNase-free, 1.5 ml elution tube, and 30 μl of Elution Buffer was added onto the membrane of the spin column. The column was centrifuged at 2,000 rpm for 2 minutes to load the membrane with the buffer, and the speed was increased to 13,000 rpm and centrifuged for 1 minute to elute the exoRNAs.

### Quantitative RT-PCR

Exo RNAs were reverse transcribed using High-Capacity cDNA Reverse Transcription Kit (ThermoFisher) in accordance with the manufacturer's instructions. To evaluate the mRNA levels of a number of genes, qRT-PCR was performed on a CFX96 qRT-PCR System using SYBR Green qRT-PCR master mix (Promega, Madison, Wis., USA). GAPDH (glyceraldehyde-3-phosphate dehydrogenase) was used as the internal control. All of the samples were normalized to internal controls, and fold changes were calculated based on relative quantification (2−ΔΔ^{Ct}).

### Example 2: Exosome Analysis

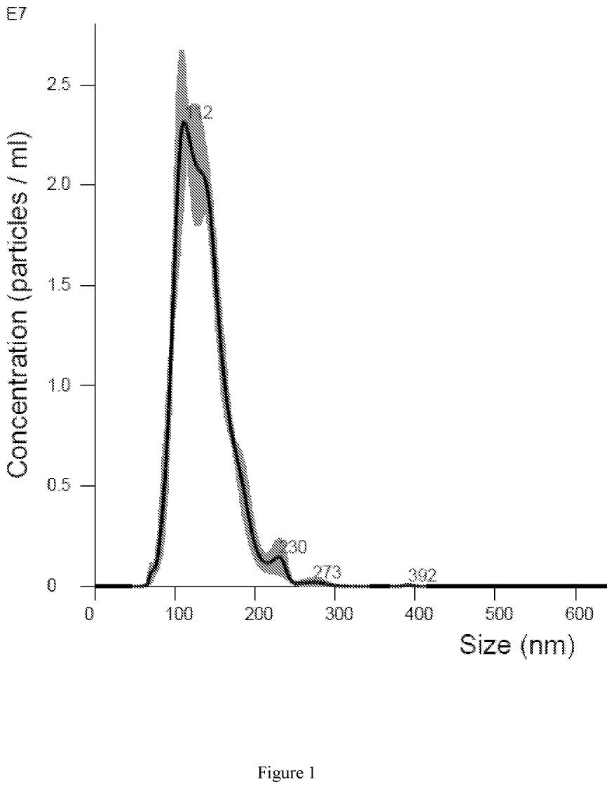

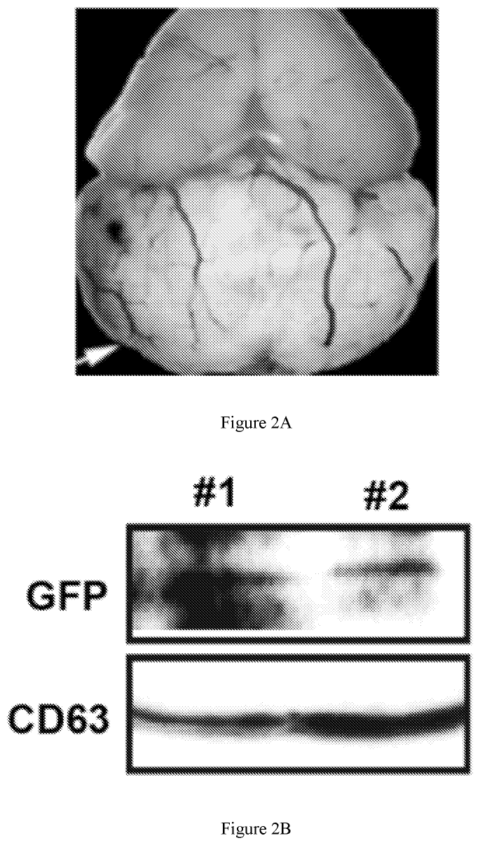

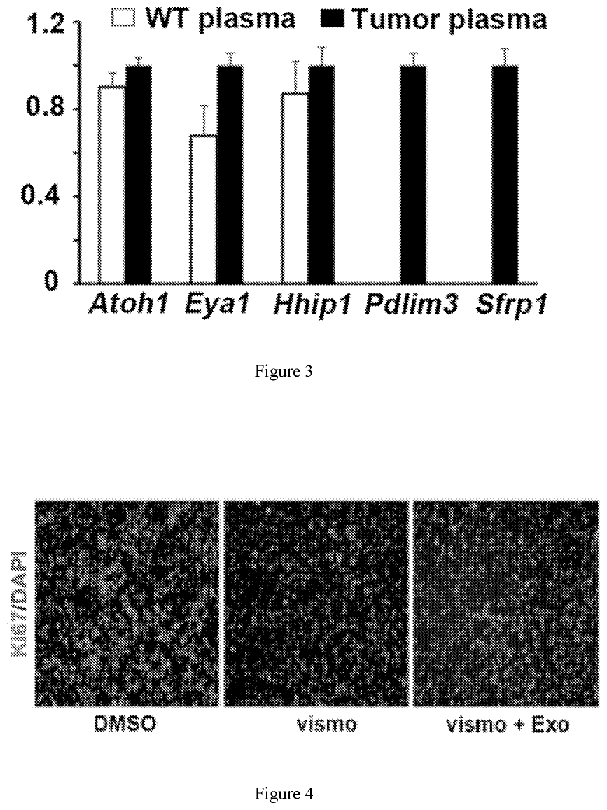

To examine whether tumor cell-derived exosomes can be found in plasma, Math1-Cre/Rosa-GFP/Ptch1^{-/-} mice were generated. In these mice, only tumor cells expressed GFP (see, FIG. 2A). Exosomes were extracted from the plasma of Math1-Cre/Rosa-GFP/Ptch1^{-/-} mice. Nanosight analysis was carried out to detect the size of exosomes yielding a size range of 50-200 nm (see, FIG. 1). GFP protein was detected in the plasma exosomes (see, FIG. 2B), suggesting that tumor cell-derived exosomes were present in the plasma. RNA was extracted from the plasma exosomes of Ptch1^{-/-} mice as well as wild type mice. Expression of Atoh1, Eya1, Hhip1, Pd-lim3 and Sfrp1 (5 gene signatures for shh group medulloblastoma) was found in plasma exosomes from both Ptch1^{-/-} mice and wild type mice, whereas Pd-lim3 and Sfrp1 mRNAs were detected only in the plasma exosomes from Ptch1^{-/-} mice (see, FIG. 3). These data suggest that plasma exosomes can be used for sub-grouping medulloblastoma.

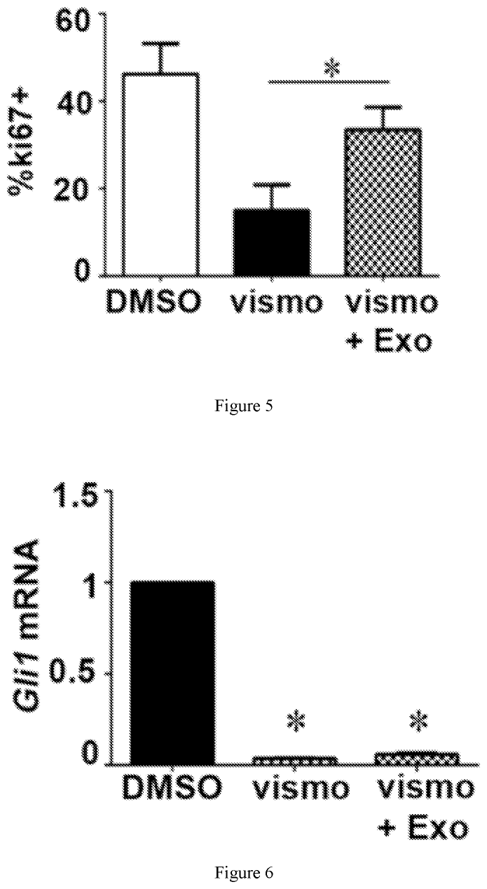

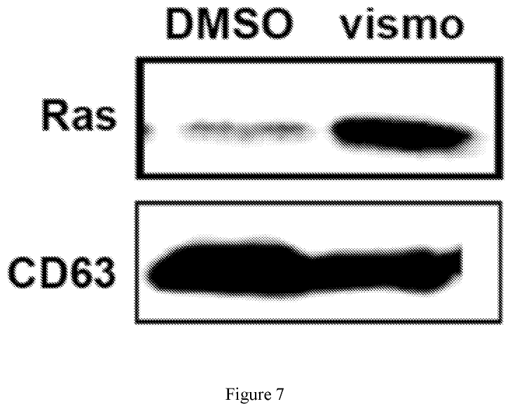

To examine the functions of exosomes in medulloblastoma cell proliferation, tumor cells from Ptch1^{-/-} mice were isolated and treated with GW4869 or DMSO for 48 hours. GW4869 significantly repressed medulloblastoma cell proliferation (data not shown), indicating that exosomes are important for medulloblastoma cell proliferation. To determine whether tumor cell-derived exosomes mediate vismodegib resistance, exosomes from medulloblastoma cells were collected and treated with 200 nM of vismodegib for 48 hours. Medulloblastoma cells from Ptch1^{-/-} mice were treated with DMSO, vismodegib, or vismodegib together with tumor cell-derived exosomes for 48 hours. Following treatment, medulloblastoma cells were harvested to examine their proliferation by immunocytochemistry (see, FIG. 4). Vismodegib significantly repressed tumor cell proliferation. However, tumor cell-derived exosomes increased medulloblastoma cell proliferation in the presence of vismodegib (see, FIG. 5). These data suggest that exosomes may mediate the resistance of medulloblastoma cells to vismodegib. Although Gli1 mRNA levels were decreased in tumor cells after vismodegib treatment, tumor cell-derived exosomes failed to upregulate Gli1 mRNA expression, indicating that exosome-stimulated proliferation in medulloblastoma cells is not driven by shh signaling (see, FIG. 6). Finally, exosomes from medulloblastoma cells were harvested and treated with DMSO or vismodegib for 48 hours. Ras protein was readily detected in exosomes derived from medulloblastoma cells treated with vismodegib. The Ras/MAPK pathway was activated in MB cells upon treatment with such exosomes (data not shown), suggesting that Ras protein in the exosome was functional. These data imply that vismodegib treatment promotes the secretion of Ras-containing exosomes in medulloblastoma cells, consistent with the role of Ras/MAPK pathway in the vismodegib resistance of tumor cells.

### Example 3: Diagnostic Significance of Exosomes in Brain Tumors

Preliminary studies revealed that exosomes derived from shh MB cells carried mRNA of shh pathway genes such as Atoh1 and Sfrp1. Moreover, tumor cell-derived exosomes can be detected in the plasma from medulloblastoma bearing mice. The dynamic changes in the mRNA and miRNA in exosomes will be further investigated in tumor cell-derived exosomes in mouse medulloblastoma at different developmental stages and of different subgroups. In addition, the mRNA, miRNA, and DNA of exosomes from plasma and CSF of patients with MB will be further examined to determine the role of plasma or CSF exosomes in subtypes and mutations of human tumor tissues.

### Examination of the Profiles of mRNA and miRNA in Exosomes from Medulloblastoma Cells and the Plasma of Mice Bearing Medulloblastoma

To examine dynamic changes of the cargo in tumor cell-derived exosomes in medulloblastoma, tumor cells from mouse shh medulloblastoma model (Ptch1^{-/-}) and group 3 medulloblastoma model (c-myc) will be collected at early stage (3 weeks of age) and late stages (8 weeks of age). As a control, cerebellar granule neuron precursors (GNPs, the normal cell of origin for medulloblastoma) will be isolated from wild type mouse brains at 1 week of age. Exosomes will be extracted from tumor cells and GNPs following the established procedure. RNAs including mRNA and miRNA will be extracted from tumor cells and GNPs as well as exosomes, which will be sequenced by Nextseq 550 Illumina system. These populations of GNPs, tumor cells (at each stage of tumor development), GNP-derived exosomes, and medulloblastoma cell-derived exosomes will be prepared for RNA sequencing. Based on mRNA and miRNA expression profiles, the alterations of exosome-carried mRNA and miRNA profiles during medulloblastoma development will be determined by DESeq2 methods. The differences in mRNA/miRNA expression profiles of tumor cells from shh medulloblastoma and group 3 medulloblastoma will be also determined by DESeq2 method. The correlation between tumor cells and their exosomes in the miRNA and mRNA profiles will be analyzed by non-parametric methods including Spearman rank correlation method. The presence of a signature mRNA and miRNA specific for tumor subgroups as well as for tumor development stages will be further validated in tumor cells and exosomes by q-PCR. In addition, the presence of a signature mRNA and miRNA will be examined in plasma exosomes harvested from mice bearing shh medulloblastoma or group 3 medulloblastoma (at 3 weeks and 6 weeks of age) as well as from wild type mice by q-PCR. A Student's t test will be used to evaluate the difference in the abundance of mRNA and miRNA, where p<0.05 will be considered as statistically significant.

These experiments are expected to identify the stage-related as well as subgroup-specific mRNAs and miRNAs that are present in tumor cell-derived exosomes and plasma exosomes of medulloblastoma mice. The dynamic changes in mRNA and miRNA in tumor cell-derived exosomes and plasma exosomes from mouse GBM models will be also analyzed. In addition, the profiles of DNA and proteins in tumor cell-derived exosomes and plasma exosomes in medulloblastoma and GBM by NGS and mass-spectrometry will be examined. These studies are expected to demonstrate the diagnostic value of exosomes in brain tumors.

### Determining the Correlation Between Exosomes in Plasma and CSF and Tumor Tissues from Medulloblastoma Patients in mRNA, miRNA and DNA Profiles

Thirty samples of plasma, CSF, and tumor tissues (before tumor treatment) from medulloblastoma patients will be acquired. Exosomes will be extracted from tumor tissue, plasma, and CSF. RNA and DNA will be extracted from tumor cells and exosomes following established protocols. Sequence profiles of RNA and DNA in tumor cells and exosomes will be generated. Medulloblastoma subgroups will be defined based on the whole transcriptome. Subtype-specific mRNA and miRNA expression in tumor cells and exosomes will be identified by DESeq2 method and correlation between tumor cells and their exosomes in CSF as well as the plasma in the mRNA and miRNA profiles will be determined. Classification of the expression profiles from exosomes will be analyzed using SVM and other predictive classification methods to identify signatures that distinguish tumor RNA profiles that can also classify RNA profiles from exosomes. Moreover, mutations in the DNA of tumor cells and exosomes will be identified. The presence of RNA and DNA in tumor cells and exosomes will be further validated by PCR. A Student's t test will be used to determine the difference of RNA and DNA levels among samples, where p<0.05 will be considered as statistically significant.

These experiments are expected to show consistency in the mRNA, miRNA, and DNA profiles between tumor tissue and exosomes in CSF as well as in plasma. Subtype-specific mRNAs and miRNAs are expected to be found in the exosomes in the CSF and plasma. In addition, DNA mutations critical for MB progression, such as the Ptch1 mutation, Smo activation, and c-myc amplification, are expected to be identified in both tumor tissue and exosomes in the CSF and plasma. The transcriptome in exosomes from CSF and plasma from MB patients after tumor treatment will be further examined to determine the correlation between transcriptomic changes in exosomes and therapeutic responsiveness and prognosis. The diagnostic value of exosomes in CSF and plasma of patients with glioblastoma multiforme will also be analyzed.

### Example 4: Therapeutic Implications of Exosomes in Brain Tumors

The preliminary studies showed that inhibition of exosome secretion by GW4869 significantly repressed medulloblastoma cell proliferation in vitro. The role of exosomes in tumor progression will be further examined by deletion of Rab27a or Rab27b (critical for exosome secretion) in tumor cells. In addition, Ras protein was detected in exosomes secreted from medulloblastoma cells after vismodegib treatment. Medulloblastoma cells became resistant to vismodegib after treatment with Ras-contained exosomes. The mechanism for secretion of Ras-containing exosomes from medulloblastoma cells following vismodegib treatment will be further examined.

### Examination of the Role of Exosome in MB Tumorigenesis by Genetic Inhibition of Exosome Secretion in Tumor Cells

To examine the role of exosomes in medulloblastoma tumorigenesis, the alterations of tumor initiation and progression after disturbing exosome secretion in medulloblastoma cells will assessed. Medulloblastoma cells isolated from Cas9/Ptch1^{-/-} mice will be infected with lentivirus carrying GFP-tagged guide RNA (sgRNA) specific for Rab27a or Rab27b or scrambled RNA for a control. Tumor cells will be harvested 48 hours after infection to examine the amount of secreted exosomes, and to analyze the proliferation and apoptosis by immunocytochemistry using antibodies against Ki67 or cleaved caspase-3. Infected cells will also be harvested to examine shh signaling by q-PCR to measure the expression Gli1 and Sfrp1. At 24 hours following infection, infected cells (GFP⁺) will be harvested and purified by FACs. 2×10⁶ GFP⁺ cells will be intracranially transplanted into CB17/SCID mice. Six CB17/SCID mice will be injected with tumor cells infected with sgRNA for Rab27a or Rab27b or control RNA (18 mice in total). When mice exhibit symptoms such as hunched back and domed head, mouse brains will be collected to examine the tumor formation by histological analyses. The incidence and latency of tumor formation will be compared by Kaplan-Meier survival curves. A one-way analysis of variance will be performed to compare all treatments with Graphpad Prism software. A 5% or lower p-value is considered to be statistically significant. In addition, recipient brains will be sectioned to examine the proliferation, differentiation, and apoptosis of tumor cells by immunohistochemistry, and activation of shh pathway in medulloblastoma cells will be analyzed by examining the expression of Gli1 and Sfrp1 by q-PCR.

These experiments are expected to show that deletion of Rab27a or Rab27b will repress medulloblastoma cell proliferation, and prohibit tumor incidence and/or prolong tumor latency in CB17/SCID mice following the transplantation. To further confirm the important functions of exosomes in medulloblastoma growth, the alterations of medulloblastoma growth in Ptch^{-/-} mice after inhibition of exosome secretion by treatment with GW4869 will be examined. The changes in transcriptome of tumor cells after deletion of Rab27a or Rab27b will be examined by RNA sequencing to determine the molecular alterations of tumor cells following inhibition of exosome secretion. In addition, the role of exosomes in glioblastoma multiforme tumorigenesis will be examined by deleting Rab27a or Rab27b in glioblastoma multiforme cells.

### Determining the Molecular Basis Underlying Exosome-Associated Resistance of MB Cells to vismodegib

Preliminary data suggest that exosome-derived Ras protein may be involved in medulloblastoma cells' resistance to vismodegib. To confirm the involvement of exosomes in vismodegib resistance of medulloblastoma cells, exosome secretion in tumor cells will be repressed by deletion of Rab27a or Rab27b. Medulloblastoma cells deficient in exosome secretion will be treated with vismodegib (200 nM). Medulloblastoma cells infected with scrambled sgRNA will be treated with vismodegib as a control. Medulloblastoma cells will be harvested 48 hours after vismodegib treatment to examine their proliferation and apoptosis by immunocytochemistry, and the shh pathway activation will be analyzed by q-PCR based on expression levels of Gli1 and Sfrp1. To further investigate whether secretion of Ras-containing exosomes in vismodegib-treated medulloblastoma cells is due to repressed shh signaling, medulloblastoma cells from Ptch1^{-/-} mice or SmoA1 mice will be isolated and treated with vismodegib (200 nM) or DMSO control for 48 hours. Exosomes will be isolated from the conditioned culture media, and Ras protein in exosomes will be examined by Western blotting. Protein levels will be quantified by Image J. The difference in the proliferation (Ki67⁺) of MB cells, as well as levels of Ras protein in exosomes, will be analyzed by Student's t test (p<0.05).

These experiments are expected to show that Rab27a or Rab27b deletion will augment the inhibition of vismodegib on the proliferation and shh signaling in tumor cells, suggesting that exosomes play a role in vismodegib resistance of tumor cells. The possible synergistic effect of vismodegib and GW4869 in inhibition of MB cell proliferation and in vivo MB growth will be examined. Vismodegib can repress shh signaling in Ptch1^{-/-} MB cells, but not in SmoA1 MB cells. Comparable levels of Ras protein in exosomes in Ptch1^{-/-} MB cells and SmoA1 MB cells following vismodegib treatment would suggest that secretion of Ras-containing exosomes from MB cells is not due to repressed shh signaling. If Ras protein could not be detected in SmoA1 MB cells after vismodegib treatment, it would indicate that vismodegib-suppressed shh signaling in tumor cells promotes the secretion of Ras-containing exosomes. The possibility of involvement Gab1 (Grb2-associated binder 1), a protein linking the shh pathway and Ras/MAPK pathway, in Ras containing exosome secretion in MB cells following vismodegib treatment will also be investigated.

### Example 5: Vismodegib and GW4869 Display Synergistic Effect in Inhibition of MB Cell Proliferation

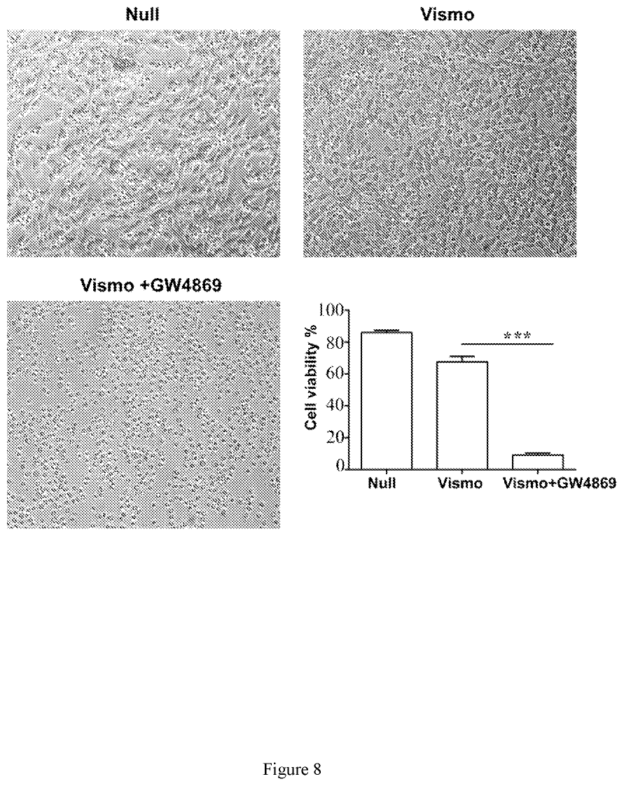

GW4869 is a commonly used pharmacological agent, which inhibits exosome generation. To examine whether vismodegib and GW4869 act synergistically in the inhibition of MB cell proliferation, MB cells were treated with the GW4869 (5 μM) and vismodegib (200 nM). Compared with Null and vismodegib inhibition of MB cells, exosome secretion significantly reduced MB cells survival (FIG. 8, ***p<0.001). These results indicate the synergistic effect of vismodegib and GW4869 in inhibition of MB cell proliferation.

### Example 6: Inhibition of Exosome Secretion Reduced MB Cell Growth After Vismodegib Treatment

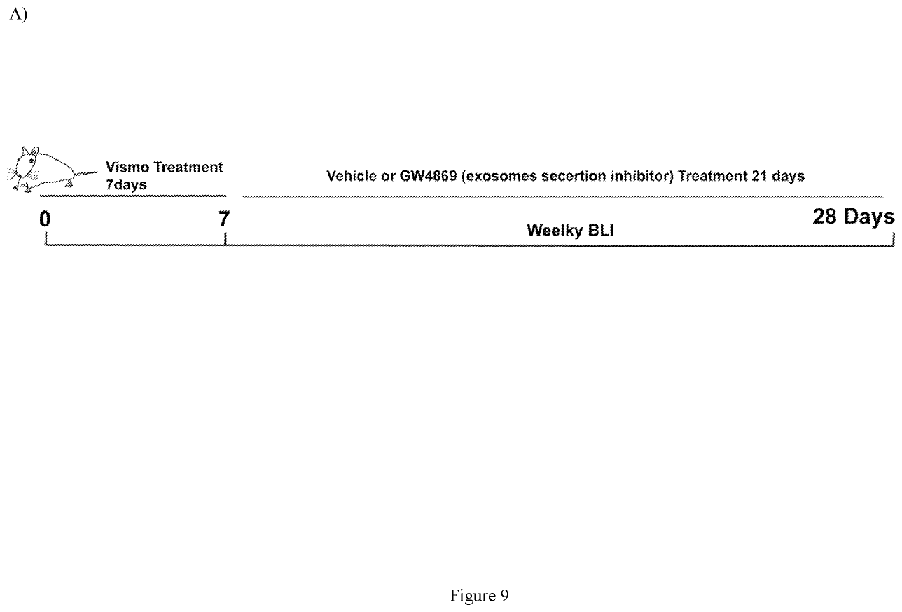

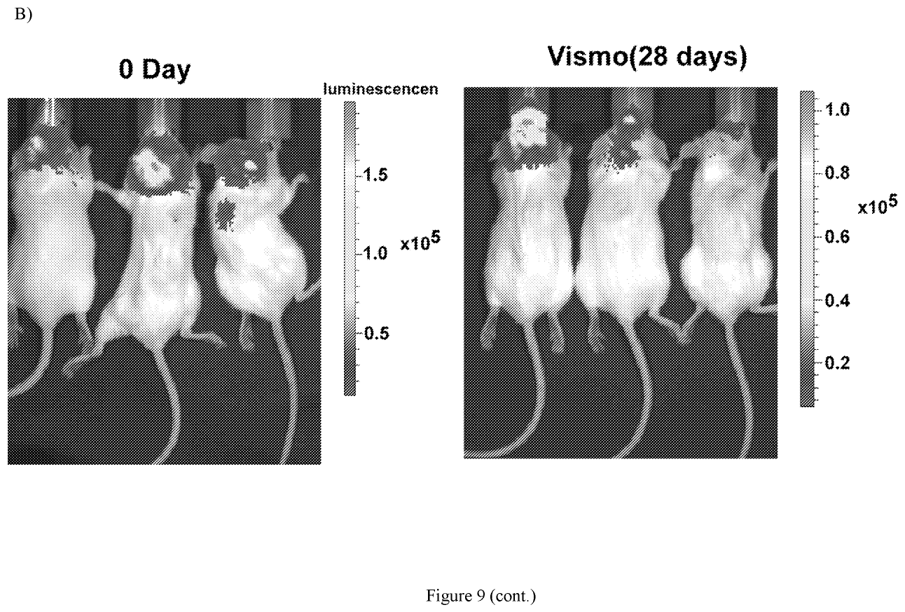

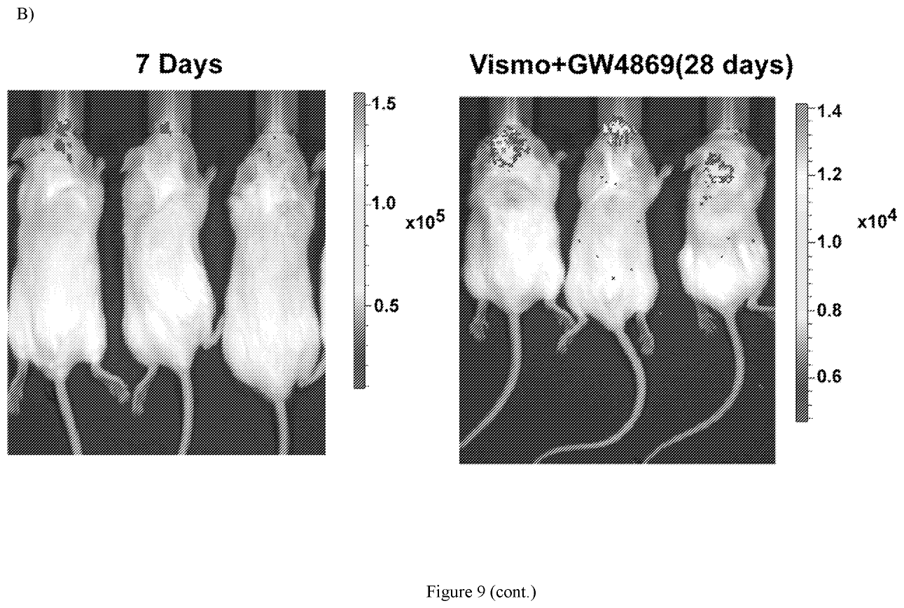

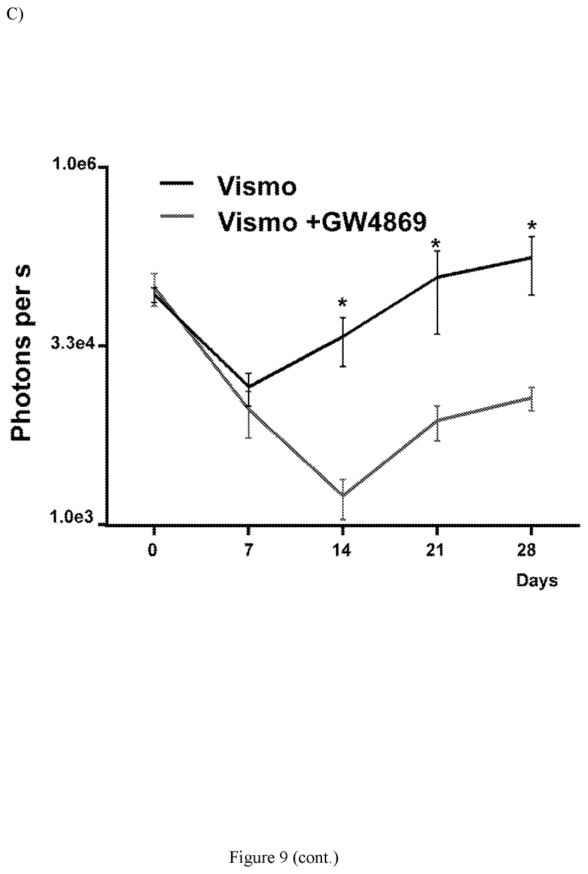

MB-luciferase cells were generated and stereotaxically injected into the cerebellum of 6- to 8-week-old NSG mice. Animals were monitored weekly using in vivo bioluminescence imaging and bioluminescence was detected as Day 0. The tumor-bearing mice were treated with vismodegib (50 mg/kg) for 7 days. These mice were separated into two groups randomly, one group was treated with a vehicle and another group was treated with GW4869 (1.25 mg/kg per day) for 3 weeks (FIG. 9A). They were monitored weekly using in vivo bioluminescence imaging (FIG. 9B). Results showed vismodegib treatment significantly reduce tumor growth. After 28 days, tumors in the two groups have a significant difference with or without GW4869 treatment (FIG. 9C, *p<0.05). These data suggest inhibition of exosomes secretion reduced MB cell growth to Hh pathway inhibitors.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of identifying a human having a brain tumor, the method comprising:
assaying the level of one or more brain tumor biomarkers in an exosomal sample obtained from the human;
comparing the level of the one or more brain tumor biomarkers in the exosomal sample from the human to the levels of the corresponding one or more brain tumor biomarkers in a reference exosomal sample, wherein an increase in the level of the one or more brain tumor biomarkers in the exosomal sample from the human compared to the reference exosomal sample indicates the presence of a sub-type of a brain tumor in the human; and
administering an anti-exosome therapeutic agent to the human.

2. The method according to claim 1, wherein the brain tumor is a glioblastoma.

3. The method according to claim 1, wherein the brain tumor is a medulloblastoma.

4. The method according to any one of claims 1 to 3, wherein the brain tumor is a Wingless (Wnt) subgroup brain tumor.

5. The method according to claim 4, wherein the brain tumor biomarker is chosen from Catenin Beta 1 (CTTNB1), Dickkopf WNT Signaling Pathway Inhibitor 1 (DKK1), Wnt Inhibitory Factor 1 (WIF1), Tenascin C (TNC), Glutamate Decarboxylase 1 (GAD1), Dickkopf WNT Signaling Pathway Inhibitor 2 (DDK2), and Empty Spiracles Homeobox 2 (EMX2).

6. The method according to claim 4, wherein the brain tumor biomarker is chosen from WIF1, TNC, GAD1, DDK2, and EMX2.

7. The method according to any one of claims 1 to 3, wherein the brain tumor is a sonic hedgehog (shh) subgroup brain tumor.

8. The method according to claim 7, wherein the brain tumor biomarker is chosen from Atonal BHLH Transcription Factor 1 (ATOH1), EYA Transcriptional Coactivator and Phosphatase 1 (EYA1), Hedgehog-Interacting Protein (HHIP), PDZ and LIM Domain Protein 3 (PDLIM3), and Secreted Frizzled-Related Protein 1 (SFRP1).

9. The method according to claim 7, wherein the brain tumor biomarker is chosen from HHIP, PDLIM3, and SFRP1.

10. The method according to any one of claims 1 to 3, wherein the brain tumor is a Group 3 subgroup brain tumor.

11. The method according to claim 10, wherein the brain tumor biomarker is chosen from Natriuretic Peptide Receptor-3 (NPR3), Interphotoreceptor Matrix Proteoglycan 2 (IMPG2), Gamma-Aminobutyric Acid Type A Receptor Alpha 5 Subunit (GABRA5), EGF-Like-Domain, Multiple 11 (EGFL11), Mab-21 Like 2 (MAB21L2), and Myc.

12. The method according to claim 10, wherein the brain tumor biomarker is chosen from NPR3, IMPG2, GABRA5, EGFL11, and MAB21L2.

13. The method according to any one of claims 1 to 3, wherein the brain tumor is a Group 4 subgroup brain tumor.

14. The method according to claim 13, wherein the brain tumor biomarker is chosen from Potassium Voltage-Gated Channel Subfamily A Member 1 (KCNA1), Eomesodermin (EOMES), KH RNA Binding Domain Containing, Signal Transduction Associated 2 (KHDRBS2), RNA Binding Motif Protein 24 (RBM24), Unc-5 Netrin Receptor D (UNC5D), and 2′-5′-Oligoadenylate Synthetase 1 (OAS1).

15. The method according to any one of claims 1 to 14, wherein the exosomal sample is a bodily fluid sample.

16. The method according to claim 15, wherein the bodily fluid is peripheral blood, sera, plasma, or cerebrospinal fluid (CSF).

17. The method according to any one of claims 1 to 16, wherein the exosomal sample comprises plasma exosomes.

18. The method according to any one of claims 1 to 16, wherein the exosomal sample comprises CSF exosomes.

19. The method according to any one of claims 1 to 18, wherein the one or more brain tumor biomarkers are mRNA biomarkers.

20. The method according to any one of claims 1 to 18, wherein the one or more brain tumor biomarkers are protein biomarkers.

21. The method according to any one of claims 1 to 18, wherein the one or more brain tumor biomarkers are miRNA biomarkers.

22. The method according to any one of claims 1 to 21, wherein the levels of the corresponding one or more brain tumor biomarkers in a reference exosomal sample comprise the average level of brain tumor biomarker expression in one or more samples from healthy, cancer-free humans.

23. The method according to any one of claims 1 to 21, wherein the levels of the corresponding one or more brain tumor biomarkers in a reference exosomal sample comprise the brain tumor biomarker expression levels in one or more exosome samples from the human obtained at an earlier timepoint.

24. The method according to any one of claims 1 to 23, wherein the exosome is isolated from the exosomal sample by size exclusion chromatography, density gradient centrifugation, differential centrifugation, nanomembrane ultrafiltration, immunoabsorbent capture, affinity purification, microfluidic separation, polymer-based precipitation, or any combination thereof.

25. The method according to any one of claims 1 to 24, wherein the anti-exosome therapeutic agent is an inhibitor of neutral sphingomyelinase and exosome biogenesis.

26. The method according to claim 25, wherein the anti-exosome therapeutic agent is GW4869 (N,N′-Bis[4-(4,5-dihydro-1H-imidazol-2-yl)phenyl]-3,3′-p-phenylene-bis-acrylamide dihydrochloride).

27. The method according to any one of claims 1 to 26, wherein the anti-exosome therapeutic agent is an inhibitor of the secretion of exosomes.

28. The method according to claim 27, wherein the anti-exosome therapeutic agent is dimethyl amiloride (DMA), neticonazole, ketoconazole, tipifarnib, isoproterenol, climbazole, triadimenol, Manumycin A, sulfisoxazole, or cannabidiol, or any combination thereof.

29. The method according to any one of claims 1 to 28, further comprising administering to the human one or more additional therapeutic agents.

30. The method according to claim 29, wherein the one or more additional therapeutic agents are chosen from a chemotherapeutic agent, a radiotherapeutic agent, an anti-angiogenic agent, a premetastatic niche formation inhibitor, and a stromal inhibitor.

31. The method according to claim 29, wherein the additional therapeutic agent is chosen from carmustine, temozolomide, bevacizumab, larotrectinib, everolimus, vincristine, lomustine, procarbazine, vismodegib, sonidegib, erlotinib, and glasdegib, or any combination thereof.

32. The method according to claim 31, wherein the therapeutic agent is chosen from vismodegib, sonidegib, and glasdegib.

33. The method according to claim 32, wherein the human is administered a combination of GW4869 or DMA with any one of vismodegib, cisplatin, and temozolomide.

34. The method according to claim 32, wherein the human is administered a combination of GW4869 and vismodegib, GW4869 and cisplatin, or GW4869 and temozolomide.

35. The method according to claim 29, wherein the additional therapeutic agent is a combination of procarbazine, lomustine, and vincristine.

36. A method of classifying a medulloblastoma tumor in a human, the method comprising:
assaying the level of one or more medulloblastoma biomarkers chosen from CTTNB1, DKK1, WIF1, TNC, GAD1, DDK2, EMX2, ATOH1, EYA1, HHIP, PDLIM3, SFRP1, NPR3, IMPG2, GABRA5, EGFL11, MAB21L2, KCNA1, EOMES, KHDRBS2, RBM24, UNC5D, and OAS1, in an exosomal sample obtained from the human;
comparing the level of the one or more biomarkers in the exosomal sample from the human to the levels of the corresponding one or more biomarkers in a reference exosomal sample, wherein:
an increase in the level of one or more of ATOH1, EYA1, HHIP, PDLIM3, and SFRP1 in the exosomal sample from the human compared to the reference exosomal sample indicates the presence of a sonic hedgehog subgroup medulloblastoma;
an increase in the level of one or more of CTTNB1, DKK1, WIF1, TNC, GAD1, DDK2, and EMX2 in the exosomal sample from the human compared to the reference exosomal sample indicates the presence of a Wnt subgroup medulloblastoma;
an increase in the level of one or more of NPR3, IMPG2, GABRA5, EGFL11, and MAB21L2 in the exosomal sample from the human compared to the reference exosomal sample indicates the presence of a Group C medulloblastoma; and
an increase in the level of one or more of KCNA1, EOMES, KHDRBS2, RBM24, UNC5D, and OAS1 in the exosomal sample from the human compared to the reference exosomal sample indicates the presence of a Group D medulloblastoma; and
administering an anti-exosome therapeutic agent to the human.

37. The method according to claim 36, wherein the medulloblastoma is a Wnt subgroup medulloblastoma.

38. The method according to claim 37, wherein the biomarker is chosen from CTTNB1, DKK1, WIF1, TNC, GAD1, DDK2, and EMX2.

39. The method according to claim 36, wherein the medulloblastoma is an shh subgroup medulloblastoma.

40. The method according to claim 39, wherein the biomarker is chosen from ATOH1, EYA1, HHIP, PDLIM3, and SFRP1.

41. The method according to claim 36, wherein the medulloblastoma is a Group 3 subgroup medulloblastoma.

42. The method according to claim 41, wherein the biomarker is chosen from NPR3, IMPG2, GABRA5, EGFL11, and MAB21L2.

43. The method according to claim 36, wherein the medulloblastoma is a Group 4 subgroup medulloblastoma.

44. The method according to claim 43, wherein the biomarker is chosen from KCNA1, EOMES, KHDRBS2, RBM24, UNC5D, and OAS1.

45. The method according to any one of claims 36 to 44, wherein the exosomal sample is a bodily fluid sample.

46. The method according to claim 45, wherein the bodily fluid is peripheral blood, sera, plasma, or CSF.

47. The method according to any one of claims 36 to 46, wherein the exosomal sample comprises plasma exosomes.

48. The method according to any one of claims 36 to 46, wherein the exosomal sample comprises CSF exosomes.

49. The method according to any one of claims 36 to 48, wherein the one or more medulloblastoma biomarkers are mRNA biomarkers.

50. The method according to any one of claims 36 to 48, wherein the one or more medulloblastoma biomarkers are protein biomarkers.

51. The method according to any one of claims 36 to 48, wherein the one or more medulloblastoma biomarkers are miRNA biomarkers.

52. The method according to any one of claims 36 to 51, wherein the levels of the corresponding one or more medulloblastoma biomarkers in a reference exosomal sample comprise the average medulloblastoma biomarker expression level in one or more exosomal samples from healthy, cancer-free humans.

53. The method according to any one of claims 36 to 51, wherein the levels of the corresponding one or more medulloblastoma biomarkers in a reference exosomal sample comprise the medulloblastoma biomarker expression level in one or more exosomal samples from the human obtained at an earlier timepoint.

54. The method according to any one of claims 36 to 51, wherein the exosome is isolated from the exosomal sample by size exclusion chromatography, density gradient centrifugation, differential centrifugation, nanomembrane ultrafiltration, immunoabsorbent capture, affinity purification, microfluidic separation, polymer-based precipitation, or any combination thereof.

55. The method according to any one of claims 36 to 54, wherein the anti-exosome therapeutic agent is an inhibitor of neutral sphingomyelinase and exosome biogenesis.

56. The method according to claim 55, wherein the anti-exosome therapeutic agent is GW4869.

57. The method according to any one of claims 36 to 54, wherein the anti-exosome therapeutic agent is an inhibitor of the secretion of exosomes.

58. The method according to claim 57, wherein the anti-exosome therapeutic agent is DMA, neticonazole, ketoconazole, tipifarnib, isoproterenol, climbazole, triadimenol, Manumycin A, sulfisoxazole, or cannabidiol, or any combination thereof.

59. The method according to any one of claims 36 to 58, further comprising administering to the human one or more additional therapeutic agents.

60. The method according to claim 59, wherein the one or more additional therapeutic agents are chosen from a chemotherapeutic agent, a radiotherapeutic agent, an anti-angiogenic agent, a premetastatic niche formation inhibitor, and a stromal inhibitor.

61. The method according to claim 59, wherein the additional therapeutic agent is chosen from carmustine, temozolomide, bevacizumab, larotrectinib, everolimus, vincristine, lomustine, procarbazine, vismodegib, sonidegib, erlotinib, and glasdegib, or any combination thereof.

62. The method according to claim 61, wherein the therapeutic agent is chosen from vismodegib, sonidegib, and glasdegib.

63. The method according to claim 62, wherein the human is administered a combination of GW4869 or DMA with any one of vismodegib, cisplatin, and temozolomide.

64. The method according to claim 62, wherein the human is administered a combination of GW4869 and vismodegib, GW4869 and cisplatin, or GW4869 and temozolomide.

65. The method according to claim 59, wherein the additional therapeutic agent is a combination of procarbazine, lomustine, and vincristine.

66. A method of treating a human having a brain tumor comprising administering to the human in need thereof an anti-exosome therapeutic agent.

67. The method according to claim 66, wherein the brain tumor is a glioblastoma.

68. The method according to claim 66, wherein the brain tumor is a medulloblastoma.

69. The method according to any one of claims 66 to 68, wherein the anti-exosome therapeutic agent is an inhibitor of neutral sphingomyelinase and exosome biogenesis.

70. The method according to claim 69, wherein the anti-exosome therapeutic agent is GW4869.

71. The method according to any one of claims 66 to 68, wherein the anti-exosome therapeutic agent is an inhibitor of the secretion of exosomes.

72. The method according to claim 71, wherein the anti-exosome therapeutic agent is DMA, neticonazole, ketoconazole, tipifarnib, isoproterenol, climbazole, triadimenol, Manumycin A, sulfisoxazole, or cannabidiol, or any combination thereof.

73. The method according to any one of claims 66 to 72, further comprising administering to the human one or more additional therapeutic agents.

74. The method according to claim 73, wherein the one or more additional therapeutic agents are chosen from a chemotherapeutic agent, a radiotherapeutic agent, an anti-angiogenic agent, a premetastatic niche formation inhibitor, and a stromal inhibitor.

75. The method according to claim 73, wherein the additional therapeutic agent is chosen from carmustine, temozolomide, bevacizumab, larotrectinib, everolimus, vincristine, lomustine, procarbazine, vismodegib, sonidegib, erlotinib, and glasdegib, or any combination thereof.

76. The method according to claim 75, wherein the therapeutic agent is chosen from vismodegib, sonidegib, and glasdegib.

77. The method according to claim 76, wherein the human is administered a combination of GW4869 or DMA with any one of vismodegib, cisplatin, and temozolomide.

78. The method according to claim 76, wherein the human is administered a combination of GW4869 and vismodegib, GW4869 and cisplatin, or GW4869 and temozolomide.

79. The method according to claim 73, wherein the additional therapeutic agent is a combination of procarbazine, lomustine, and vincristine.

80. The method according to any one of claims 66 to 79, wherein the human is determined to have the brain tumor by a method comprising:
assaying the level of one or more brain tumor biomarkers chosen from CTTNB1, DKK1, WIF1, TNC, GAD1, DDK2, EMX2, ATOH1, EYA1, HHIP, PDLIM3, SFRP1, NPR3, IMPG2, GABRA5, EGFL11, MAB21L2, KCNA1, EOMES, KHDRBS2, RBM24, UNC5D, and OAS1, in an exosomal sample obtained from the human; and
comparing the level of the one or more biomarkers in the exosomal sample from the human to the levels of the corresponding one or more biomarkers in a reference exosomal sample, wherein:
an increase in the level of one or more of ATOH1, EYA1, HHIP, PDLIM3, and SFRP1 in the exosomal sample from the human compared to the reference exosomal sample indicates the presence of a sonic hedgehog subgroup brain tumor;
an increase in the level of one or more of CTTNB1, DKK1, WIF1, TNC, GAD1, DDK2, and EMX2 in the exosomal sample from the human compared to the reference exosomal sample indicates the presence of a Wnt subgroup brain tumor;
an increase in the level of one or more of NPR3, IMPG2, GABRA5, EGFL11, and MAB21L2 in the exosomal sample from the human compared to the reference exosomal sample indicates the presence of a Group C brain tumor; and
an increase in the level of one or more of KCNA1, EOMES, KHDRBS2, RBM24, UNC5D, and OAS1 in the exosomal sample from the human compared to the reference exosomal sample indicates the presence of a Group D brain tumor.

81. The method according to claim 80, wherein the exosomal sample is a bodily fluid sample.

82. The method according to claim 81, wherein the bodily fluid is peripheral blood, sera, plasma, or CSF.

83. The method according to any one of claims 80 to 82, wherein the exosomal sample comprises plasma exosomes.

84. The method according to any one of claims 80 to 82, wherein the exosomal sample comprises CSF exosomes.

85. The method according to any one of claims 80 to 84, wherein the one or more brain tumor biomarkers are mRNA biomarkers.

86. The method according to any one of claims 80 to 84, wherein the one or more brain tumor biomarkers are protein biomarkers.

87. The method according to any one of claims 80 to 84, wherein the one or more brain tumor biomarkers are miRNA biomarkers.

88. The method according to any one of claims 80 to 87, wherein the levels of the corresponding one or more brain tumor biomarkers in a reference exosomal sample comprise the average brain tumor biomarker expression level in one or more exosomal samples from healthy, cancer-free humans.

89. The method according to any one of claims 80 to 87, wherein the levels of the corresponding one or more brain tumor biomarkers in a reference exosomal sample comprise the brain tumor biomarker expression level in one or more exosomal samples from the human obtained at an earlier timepoint.

90. The method according to any one of claims 80 to 89, wherein the exosome is isolated from the exosomal sample by size exclusion chromatography, density gradient centrifugation, differential centrifugation, nanomembrane ultrafiltration, immunoabsorbent capture, affinity purification, microfluidic separation, polymer-based precipitation, or any combination thereof.

91. A method of suppressing vismodegib resistance in a human having a vismodegib-resistant brain tumor, the method comprising administering to the human in need thereof an anti-exosome therapeutic agent.

92. The method according to claim 91, wherein the brain tumor is a glioblastoma.

93. The method according to claim 91, wherein the brain tumor is a medulloblastoma.

94. The method according to any one of claims 91 to 93, wherein the anti-exosome therapeutic agent is an inhibitor of neutral sphingomyelinase and exosome biogenesis.

95. The method according to claim 94, wherein the anti-exosome therapeutic agent is GW4869.

96. The method according to any one of claims 91 to 93, wherein the anti-exosome therapeutic agent is an inhibitor of the secretion of exosomes.

97. The method according to claim 96, wherein the anti-exosome therapeutic agent is DMA, neticonazole, ketoconazole, tipifarnib, isoproterenol, climbazole, triadimenol, Manumycin A, sulfisoxazole, or cannabidiol, or any combination thereof.

98. The method according to any one of claims 91 to 97, further comprising administering to the human one or more additional therapeutic agents.

99. The method according to claim 98, wherein the one or more additional therapeutic agents are chosen from a chemotherapeutic agent, a radiotherapeutic agent, an anti-angiogenic agent, a premetastatic niche formation inhibitor, and a stromal inhibitor.

100. The method according to claim 98, wherein the additional therapeutic agent is chosen from carmustine, temozolomide, bevacizumab, larotrectinib, everolimus, vincristine, lomustine, procarbazine, vismodegib, sonidegib, erlotinib, and glasdegib, or any combination thereof.

101. The method according to claim 100, wherein the therapeutic agent is chosen from vismodegib, sonidegib, and glasdegib.

102. The method according to claim 101, wherein the human is administered a combination of GW4869 or DMA with any one of vismodegib, cisplatin, and temozolomide.

103. The method according to claim 101, wherein the human is administered a combination of GW4869 and vismodegib, GW4869 and cisplatin, or GW4869 and temozolomide.

104. The method according to claim 98, wherein the additional therapeutic agent is a combination of procarbazine, lomustine, and vincristine.

105. A method of monitoring brain tumor treatment in a human comprising:
assaying the level of one or more brain tumor biomarkers in a first exosomal sample obtained from the human and a second exosomal sample obtained from the human, wherein the second exosomal sample is obtained from the human after the first exosomal sample; and
comparing the level of the one or more brain tumor biomarkers in the first exosomal sample to the level of the one or more brain tumor biomarkers in the second exosomal sample, wherein:
a decrease in the level of the one or more brain tumor biomarkers in the second exosomal sample compared to the first exosomal sample indicates the human is responding favorably to the brain tumor treatment; and
no change or an increase in the level of the one or more brain tumor biomarkers in the second exosomal sample compared to the first exosomal sample indicates the human is not responding favorably to the brain tumor treatment.

106. The method according to claim 105, wherein the first exosomal sample is obtained from the human prior to initiation of treatment and the second exosomal sample is obtained from the human after initiation of treatment.

107. The method according to claim 106, wherein the first exosomal sample is obtained from the human after the human is diagnosed with the brain tumor and before the initiation of treatment, and the second exosomal sample is obtained from the human within one month after the initiation of treatment.

108. The method according to any one of claims 105 to 107, wherein the brain tumor is a glioblastoma.

109. The method according to any one of claims 105 to 107, wherein the brain tumor is a medulloblastoma.

110. The method according to any one of claims 105 to 109, wherein the brain tumor is a Wnt subgroup brain tumor.

111. The method according to claim 110, wherein the brain tumor biomarker is chosen from CTTNB1, DKK1, WIF1, TNC, GAD, DDK2, and EMX2.

112. The method according to any one of claims 105 to 109, wherein the brain tumor is an shh subgroup brain tumor.

113. The method according to claim 112, wherein the brain tumor biomarker is chosen from ATOH1, EYA1, HHIP, PDLIM3, and SFRP1.

114. The method according to any one of claims 105 to 109, wherein the brain tumor is a Group 3 subgroup brain tumor.

115. The method according to claim 114, wherein the brain tumor biomarker is chosen from NPR3, IMPG2, GABRA5, EGFL11, and MAB21L2.

116. The method according to any one of claims 105 to 109, wherein the brain tumor is a Group 4 subgroup brain tumor.

117. The method according to claim 116, wherein the brain tumor biomarker is chosen from KCNA1, EOMES, KHDRBS2, RBM24, UNC5D, and OAS1.

118. The method according to any one of claims 105 to 117, wherein the exosomal sample is a bodily fluid sample.

119. The method according to claim 118, wherein the bodily fluid is peripheral blood, sera, plasma, or CSF.

120. The method according to any one of claims 105 to 119, wherein the exosomal sample comprises plasma exosomes.

121. The method according to any one of claims 105 to 119, wherein the exosomal sample comprises CSF exosomes.

122. The method according to any one of claims 105 to 121, wherein the one or more brain tumor biomarkers are mRNA biomarkers.

123. The method according to any one of claims 105 to 121, wherein the one or more brain tumor biomarkers are protein biomarkers.

124. The method according to any one of claims 105 to 121, wherein the one or more brain tumor biomarkers are miRNA biomarkers.

125. The method according to any one of claims 105 to 124, wherein the exosome is isolated from the exosomal sample by size exclusion chromatography, density gradient centrifugation, differential centrifugation, nanomembrane ultrafiltration, immunoabsorbent capture, affinity purification, microfluidic separation, polymer-based precipitation, or any combination thereof.

126. The method according to any one of claims 105 to 125, wherein the brain tumor treatment is chemotherapy, radiotherapy, anti-angiogenic therapy, or surgery.

127. The method according to any one of claims 105 to 126, further comprising modifying the course of treatment for the human when there is no change or an increase in the level of the one or more brain tumor biomarkers in the second exosomal sample compared to the first exosomal sample.

128. The method according to claim 127, wherein the modification of treatment comprises administering an anti-exosome therapeutic agent to the human.

129. The method according to claim 128, wherein the anti-exosome therapeutic agent is an inhibitor of neutral sphingomyelinase and exosome biogenesis.

130. The method according to claim 129, wherein the anti-exosome therapeutic agent is GW4869.

131. The method according to claims 128, wherein the anti-exosome therapeutic agent is an inhibitor of the secretion of exosomes.

132. The method according to claim 128, wherein the anti-exosome therapeutic agent is DMA, neticonazole, ketoconazole, tipifarnib, isoproterenol, climbazole, triadimenol, Manumycin A, sulfisoxazole, or cannabidiol, or any combination thereof.

133. The method according to claim 128, wherein the modification of treatment comprises administering a combination of GW4869 or DMA with any one of vismodegib, cisplatin, and temozolomide to the human.

134. The method according to claim 133, wherein the human is administered a combination of GW4869 and vismodegib, GW4869 and cisplatin, or GW4869 and temozolomide.

135. Anti-exosome therapeutic agents for use in treating a human having a brain tumor.

136. Anti-exosome therapeutic agents for use in the preparation of a medicament for treating a human having a brain tumor.

137. Anti-exosome therapeutic agents for use in suppressing vismodegib resistance in a human having a vismodegib-resistant brain tumor.

138. Anti-exosome therapeutic agents for use in the preparation of a medicament for suppressing vismodegib resistance in a human having a vismodegib-resistant brain tumor.
